(19) United States
(12) Patent Application Publication
(10) Pub. No.: US 2006/0035810 A1
(43) Pub. Date: Feb. 16, 2006

(54) Regulation of ins(3456)p4 signalling by a reversible kinase phosphatase and methods and compositions related thereto

(76) Inventors: Stephen B Shears, Chapel Hill, NC (US); Barry V.L Potter, Bath (GB); Andrew M Riley, Bath (GB)

Correspondence Address: NATIONAL INSTITUTE OF HEALTH;C/O NEEDLE & ROSENBERG, P.C., SUITE 1000, 999 PEACHTREE STREET, ATLANTA, GA, 30303, US

(21) Appl. No.: 10/508,363

(22) PCT Filed: Mar. 18, 2003
(86) PCT No.: PCT/US03/08641

§ 371 Date: Jun. 30, 2005

Related U.S. Application Data
(60) Provisional application No. 60/365,258, filed on Mar. 18, 2002.

(51) Int. Cl. A61K 31/66

(52) U.S. Cl. 514/1; 514/102

(57) ABSTRACT

Provided is a method of increasing 3,4,5,6-tetrakisphosphate by increasing the activity of inositol 1,3,4,5,6 pentakisphosphate 1-phosphatase, and a method of decreasing 3,4,5,6-tetrakisphosphate by decreasing the activity of inositol 1,3,4,5,6 pentakisphosphate 1-phosphatase. A method of reducing salt, fluid or mucous secretion in a subject, comprising increasing the activity of inositol 1,3,4,5,6 pentakisphosphate 1-phosphatase in the subject is provided. A method of treating a disease that is exacerbated by salt, fluid or mucous secretion is also provided, comprising increasing the activity of inositol 1,3,4,5,6 pentakisphosphate phosphatase in a subject having a disease that is exacerbated by mucous, whereby mucous secretion is reduced and the disease is treated. Also provided is method of increasing salt and fluid secretion in a subject, comprising decreasing the activity of inositol 1,3,4,5,6 pentakisphosphate 1-phosphatase in the subject. A method of treating a disease that is treated by increased salt and fluid secretion is provided, comprising decreasing the activity of inositol 1,3,4,5,6 pentakisphosphate 1-phosphatase in a subject having a disease that is treated by increased salt and fluid secretion, whereby salt and fluid secretion is increased and the disease is treated.

### CROSS-REFERENCE TO RELATED APPLICATIONS

This application claims the benefit of priority of U.S. Provisional Application No. 60/365,258, filed Mar. 18, 2002, which is hereby incorporated in its entirety herein by reference.

### FIELD OF THE INVENTION

This invention relates generally to control of chloride channel conductance by Ins(3,4,5,6)P₄.

### BACKGROUND

Regulation of Cl⁻ channel conductance by Ins(3,4,5,6)P₄ provides receptor-dependent control over salt and fluid secretion (Carew et al., “Ins(3,4,5,6)P₄ inhibits an apical calcium-activated chloride conductance in polarized monolayers of a cystic fibrosis cell-line,” J Biol Chem 2000, 275: 26906-26913), cell-volume homeostasis (Nilius et al., “Inhibition by inositoltetrakisphosphates of calcium- and volume-activated Cl⁻ currents in macrovascular endothelial cells,” Pflügers Arch—Eur J Physiol 435: 637-644 (1998)), and electrical excitability of neurons and smooth muscle (Ho et al., “Regulation of chloride channel conductance by Ins(3,4,5,6)P₄; a phosphoinositide-initiated signalling pathway that acts downstream of Ins(1,4,5)P₃,” In Frontiers in Molecular Biology: Biology of Phosphoinositides. Edited by Cockroft S. Oxford: Oxford University Press; 2000:298-319)). Ignorance of how Ins(3,4,5,6)P₄ is synthesized has long hindered the understanding of this signalling pathway.

We now show Ins(3,4,5,6)P₄ synthesis by Ins(1,3,4,5,6)P₅ 1-phosphatase activity, by an enzyme previously characterized (Yang et al., “Multitasking in Signal Transduction by a Promiscuous Human Ins(3,4,5,6)P₄ 1-Kinase/Ins(1,3,4)P₃ 5/6Kinase,” Biochem J. 351: 551-555 (2000)) as an Ins(3,4,5,6)P₄ 1-kinase. Rationalization of these phenomena with a ligand-binding model unveils Ins(1,3,4)P₃ as not simply an alternative kinase substrate (Yang et al., “Multitasking in Signal Transduction by a Promiscuous Human Ins(3,4,5,6)P₄ 1-Kinase/Ins(1,3,4)P₃ 5/6-Kinase,” Biochem J. 351: 551-555 (2000); Wilson et al., “Isolation of inositol 1,3,4-trisphosphate 5/6-kinase, cDNA cloning, and expression of recombinant enzyme,” J Biol. Chem. 271: 11904-11910 (1996)), but also an activator of Ins(1,3,4,5,6)P₅ 1-phosphatase.

Further, we also show that a second class of Ins(3,4,5,6)P₄-regulated chloride channels in intracellular vesicles regulate insulin secretion and the release of certain neurotransmitters. These physiological processes, like the secretion in epithelial cells, contraction in smooth muscle and nerve impulse transmission in neurons, can be modulated by genetic and pharmacologic manipulation of the phosphatase that synthesizes Ins(3,4,5,6)P₄.

### SUMMARY OF THE INVENTION

In accordance with the purpose(s) of this invention, as embodied and broadly described herein, this invention, in one aspect, relates to a method of increasing 3,4,5,6-tetrakisphosphate by increasing the activity of inositol 1,3,4,5,6 pentakisphosphate 1-phosphatase, and in another aspect relates to a method of decreasing 3,4,5,6-tetrakisphosphate by decreasing the activity of inositol 1,3,4,5,6 pentakisphosphate 1-phosphatase.

A method of reducing salt, fluid or mucous secretion in a subject, comprising increasing the activity of inositol 1,3,4,5,6 pentakisphosphate 1-phosphatase in the subject is provided.

A method of treating a disease that is exacerbated by salt, fluid or mucous secretion is also provided, comprising increasing the activity of inositol 1,3,4,5,6 pentakisphosphate 1-phosphatase in a subject having a disease that is exacerbated by salt fluid or mucous secretion, whereby salt, fluid or mucous secretion is reduced and the disease is treated. Any disease exacerbated by salt, fluid or mucous secretion is a target of the invention, with specific examples including asthma, bronchitis and the common cold.

Also provided is method of increasing salt or fluid secretion in a subject, comprising decreasing the activity of inositol 1,3,4,5,6 pentakisphosphate 1-phosphatase in the subject.

A method of treating a disease that is treated by increased salt or fluid secretion is provided, comprising decreasing the activity of inositol 1,3,4,5,6 pentakisphosphate 1-phosphatase in a subject having a disease that is treated by increased salt or fluid secretion, whereby salt or fluid secretion is increased and the disease is treated. Any of the currently available or later-developed methods for decreasing the activity of this enzyme are contemplated to be within the scope of the invention. Cystic fibrosis is one example of the diseases that could be treated by increased mucous secretion.

A method of treating a disease that is treated by increased inositol 1,3,4,5,6 pentakisphosphate 1-phosphatase activity in a subject having a disease that is treated by decreased insulin granule acidification and concomitant insulin secretion, whereby insulin secretion is decreased and the disease is treated.

A method of treating a disease that is treated by decreased inositol 1,3,4,5,6 pentakisphosphate 1-phosphatase activity in a subject having a disease that is treated by increased insulin granule cell acidification or maintenance of acidification, thereby allowing or promoting insulin secretion, whereby insulin secretion is maintained at an adequate and desired level and the disease is treated. Type 2 diabetes is one example of the diseases that could be treated by allowing or promoting insulin secretion.

A method of decreasing chloride secretion from calcium-activated chloride channels in a cell, comprising decreasing inositol 3,4,5,6-phosphate kinase activity in the cell is provided. A method of decreasing chloride secretion from calcium-activated chloride channels in a cell, comprising increasing inositol 1,3,4,5,6 pentakisphosphate 1-phosphatase activity in the cell is also provided. The methods of decreasing chloride secretion can be in a cell selected from the group consisting of an epithelial cell, a neuron and a smooth muscle cell.

A method of increasing chloride secretion from calcium-activated chloride channels in a cell, comprising increasing inositol 3,4,5,6 phosphate kinase activity in the cell is provided. Also provided is a method of increasing the activity of calcium-activated chloride channels in a cell, comprising decreasing inositol 1,3,4,5,6 pentakisphosphate 1-phosphatase activity in the cell. The methods of increasing chloride secretion can be in a cell selected from the group consisting of an epithelial cell, a neuron and a smooth muscle cell.

A method of reducing inositol 3,4,5,6 tetrakisphosphate 1-phosphate-mediated inhibition of insulin release is provided. This method includes decreasing the activity of inositol 1,3,4,5,6 pentakisphosphate 1-phosphatase in the subject.

A method of treating a disease that is exacerbated by inhibition of insulin secretion is also provided that includes decreasing the activity of inositol 1,3,4,5,6 pentakisphosphate 1-phosphatase in a subject having a disease that is exacerbated by inhibition of insulin secretion, whereby the inhibition of insulin secretion is reduced and the disease is treated.

A method of increasing inositol 3,4,5,6 tetrakisphosphate 1-phosphate-mediated inhibition of insulin release, comprising increasing the activity of inositol 1,3,4,5,6 pentakisphosphate 1-phosphatase in the subject is provided.

A method of treating a disease that is treated by increasing levels of inositol 3,4,5,6 tetrakisphosphate thereby modulating inositol 3,4,5,6 tetrakisphosphate 1-phosphate-regulated chloride channels in intracellular vesicles, comprising altering the activity of inositol 1,3,4,5,6 pentakisphosphate 1-phosphatase in a subject having a disease that is treated by decreased inositol 1,3,4,5,6 pentakisphosphate 1-phosphatase activity, whereby the disease is treated is provided.

A method of treating a disease that is treated by decreasing levels of inositol 3,4,5,6 tetrakisphosphate thereby modulating inositol 3,4,5,6 tetrakisphosphate 1-phosphate-regulated chloride channels in intracellular vesicles, comprising altering the activity of inositol 1,3,4,5,6 pentakisphosphate 1-phosphatase in a subject having a disease that is treated by increased inositol 1,3,4,5,6 pentakisphosphate 1-phosphatase activity, whereby the disease is treated is provided.

A method of decreasing chloride secretion from calcium-activated chloride channels in an intracellular vesicle, comprising decreasing inositol 3,4,5,6 phosphate kinase activity in the cell is provided.

A method of increasing chloride secretion from calcium-activated chloride channels in an intracellular vesicle, comprising increasing inositol 3,4,5,6 phosphate kinase activity in the intracellular vesicle is provided.

A method of increasing the activity of calcium-activated chloride channels in an intracellular vesicle, comprising decreasing inositol 1,3,4,5,6 pentakisphosphate 1-phosphatase activity in the intracellular vesicle is provided.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate some embodiment(s) of the invention and together with the description, serve to explain the principles of the invention.

FIG. 1 shows ADP-dependent dephosphorylation of Ins(1,3,4,5)P₄, Ins(1,3,4,6)P₄ and Ins(1,3,4,5,6)P₅. Ins(3,4,5,6)P₄ kinase (0.4 μg) was incubated for 30 min in 100 μl phosphatase assay buffer (see Methods) either with (closed circles) or without (open circles) 5 mM ADP plus trace-amounts of either [³H]Ins(1,3,4,5,6)P₅ (panel A) [³H]Ins(1,3,4,6)P₄ (panel B), or [³H]Ins(1,3,4,5)P₄ (panel C). Samples were analyzed by HPLC (see Methods). The elution positions of Ins(1,3,4)P₃ and Ins(3,4,5,6)P₄ were verified with genuine standards. The elution positions of Ins(1,3,4,6)P₄ (106 min, panel B) and of Ins(1,3,4,5)P₄ (110 min, panel C) exclude these as products of InsP₅ dephosphorylation (panel A).

FIG. 2 shows the proposed kinase/phosphatase reaction pathway. The schematic shows how three proposed substrate-binding modes for the enzyme are employed to permit interconversion of Ins(1,3,4)P₃, Ins(3,4,6)P₃, Ins(1,3,4,5)P₄ and Ins(1,3,4,6)P₄; Ins(3,4,5,6)P₄/s(1,3,4,5,6)P₅ interconversion is also shown separately. All of these inositol phosphates are shown bound to the enzyme (depicted as a reverse “L”), although the switch from one binding mode to another may involve release and re-binding of an inositol phosphate by the enzyme. If a single inositolphosphate binding site is assumed, then the placement of certain hydroxyl and phosphate (‘P’) groups (colored red) is equivalent in all three binding modes. The group subject to reversible phosphorylation/dephosphorylation is highlighted in orange.

FIG. 3 shows HPLC Analysis of the products of Ins(3,4,6)P₃ phosphorylation by the Ins(3,4,5,6)P₄ kinase. Ins(3,4,5,6)P₄ kinase (3.4 μg) was incubated in 200 μl kinase assay buffer for 0 (dotted trace) or 4 hr. (solid trace) with 50 μM Ins(3,4,6)P₃ as described under Methods. Samples were analyzed by a mass detection HLPC technique (see Methods). The absorbance at the 30 min elution time was arbitrarily set to zero. Elution positions of Ins(2,3,4,6)P₄ and Ins(3,4,5,6)P₄ are shown. Identical results were obtained with a second, independently synthesized source of Ins(3,4,6)P₃ (see Methods).

FIG. 4 shows that Ins(1,3,4)P₃ activates Ins(1,3,4,5,6)P₅ 1-phosphatase activity. Panel A shows the degree of 1-phosphatase activity towards 5 μM [³H]Ins(1,3,4,5,6)P₅ in 15 min assays performed as described in the legend to FIG. 1; reactions were supplemented with either 0, 1 or 5 μM Ins(1,3,4)P₃ plus 0.06 μg enzyme (means±SE, n=5). Additional incubations were performed with 5 μM [³H]Ins(1,3,4)P₃ (approx. 1200 d.p.m.) plus either no Ins(1,3,4,5,6)P₅ (open circles) or 5 μM non-radiolabeled Ins(1,3,4,5,6)P₅ (closed circles); these reactions were assayed by HPLC (Synchropak Q100 column) and the InsP₄ region of the chromatogram is shown in Panel B.

FIG. 5 shows over-expression of InsP₅ 1-phosphatase activity in T₈₄ cells. Panel A: Western blotting of the FLAG epitope in aliquots of cell lysates from vector (V, 20 μl) and enzyme (E, 10 μl) transfected T₈₄ cells. Mol. Wt. markers are given. Panels B and C respectively show levels of [³H]Ins(3,4,5,6)P₄ and [³H]InsP₅ in vector- and enzyme-transfected T₈₄ cells, pre-labeled with 50 μCi [³H]inositol/ml for 4 days, and incubated for 15 min with either vehicle or 100 μM carbachol (CCh). Data were normalized to the level of [³H]PtdIns. The asterisk denotes that levels in vector-transfected cells were significantly (p<0.02, n=4, paired t test) lower than those in enzyme-transfected cells. Panel D shows Cl⁻ secretion (assayed as short-circuit current, I_{SC}) across a T₈₄ monolayer in response to 100 μM carbachol added to the basolateral surface. Data are composite curves from 12 experiments with vector- and enzyme-transfected cells.

FIG. 6. Ins(3,4,5,6)P₄ inhibits acidification of insulin granules in pancreatic β-cells. FIG. 6A, Representative time-courses for intragranular acidification, monitored by increased fluorescence of LSG before and after establishment of the standard whole-cell configuration (see arrow) and the effect of perfusion of intracellular medium (containing 0.4 μM [Ca²⁺]_{free} except where “no calcium” is indicated; see Methods) plus either 0 (“control”), 1 or 10 μM Ins(3,4,5,6)P₄. FIG. 6B, Mean (±SE) fluorescence changes (n-5-11), during the 60 s that followed establishment of the standard whole-cell configuration, for cells perfused with inositol phosphates as indicated at the base of the bar graph. Where indicated, DIDS (100 μM) were added to the bath solution 30 min prior to patching the cells. Statistical significance was evaluated by Student's unpaired t-test (* P<0.01).

FIG. 7 shows data that Ins(3,4,5,6)P₄ inhibits CCCP-mediated de-acidification of insulin granules. FIG. 7A, Representative confocal images of LSG fluorescence immediately before (zero) and 60 s after perfusion of cells with intracellular buffer supplemented with 100 μM of the protonophore, CCCP, in the absence or presence of 10 μM Ins(3,4,5,6)P₄. FIG. 7B, Representative time-courses for changes in LSG-fluorescence ([F/F₀] as % of zero time) immediately after establishing a standard whole-cell configuration as in panel A, with intracellular medium (containing 0.4 μM [Ca²⁺]_{free} except where “no calcium” is indicated; see Methods) plus 100 μM CCCP, together with either 0 (“control”) or 10 μM Ins(3,4,5,6)P₄. FIG. 7C, Mean (±SE) fluorescence changes (n=4-6), during the 60 s that followed establishment of the standard whole-cell configuration, for cells perfused with intracellular medium supplemented with inositol phosphates as indicated at the base of the bar graph (* P<0.0).

FIG. 8 shows data indicating that Ins(3,4,5,6)P₄-dependent inhibition of Ca²⁺-induced α-cell exocytosis is conditional upon [Ca²⁺]_{free}. FIG. 8A, Representative increases in cell capacitance (ΔC), reflecting exocytosis (as is described in Barg et al., J. Cell Sci 114: 2145-2154 (2001)), elicited by intracellular perfusion with intracellular solution (0.4 μM [Ca²⁺]_{free}), containing either 0 (“control”) or 10 μM Ins(3,4,5,6)P₄. FIG. 8B, Average rates of capacitance increase (ΔC/Δt) during the first minute of recording, as described for FIG. 8A, in 5-17 experiments. FIG. 8C, Representative increases in ΔC, measured under the same conditions as in FIG. 8A, except that the [Ca²⁺]_{free} was 1.5 μM. FIG. 8D, Average rates of capacitance increase (ΔC/Δt) during the first minute of recording, as described for FIG. 8C, in 6-11 experiments.

### DESCRIPTION OF THE PREFERRED EMBODIMENTS

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific inhibitors or activators, specific enzyme substrates, or to particular expression constructs as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a compound” includes mixtures of compounds, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

The following abbreviations have been used: CCCP, carbonyl cyanide m-chlorophenylhydrazone, CLCA, calcium-activated chloride channel; Ins(1,4,5)P₃, inositol 1,4,5-trisphosphate; DIDS, 4,4′-diisothiocyanostilbene-2,2′-disulfonic acid; Ins(3,4,5,6)P₄, inositol 3,4,5,6-tetrakisphosphate; Ins(1,4,5,6)P₄, inositol 1,4,5,6-tetrakisphosphate; Ins(1,3,4,5,6)P₅, inositol 1,3,4,5,6-pentakisphosphate, LSG, LysoSensor-Green™ DND-189.

Reference will now be made in detail to the present preferred embodiments of the invention, examples of which are illustrated in the accompanying drawings. Wherever possible, the same reference numbers are used throughout the drawings to refer to the same or like parts.

A method of reducing salt, fluid or mucous secretion in a subject is provided, comprising increasing the activity of inositol 1,3,4,5,6-pentakisphosphate 1-phosphatase in the subject. The currently available or later-developed methods and compounds for increasing the activity of this enzyme are contemplated to be within the scope of the invention. Examples of such compounds and methods are described herein and in other art that is incorporated by reference herein.

A method of modulating the secretion of insulin and neurotransmitter release in a subject is also provided that comprises increasing, decreasing or preventing the increase or decrease of activity of inositol 1,3,4,5,6-pentakisphosphate 1-phosphatase in a subject. In the practice of this method, a class of Ins(3,4,5,6)P₄-regulated chloride channels in intracellular vesicles is manipulated thereby providing for increased or decreased insulin secretion or preventing or facilitating the regulation of insulin secretion in response to other metabolic, hormonal or physiological signals. This method can be used for the treatment or alleviation of symptoms of type 2 diabetes or similar types of diseases or disorders.

The enzyme that is the target of the present methods was originally identified by the inventors as inositol 1,3,4-trisphosphate 5/6-kinase. Later, it was discovered the enzyme was also an inositol 3,4,5,6-tetrakisphosphate 1-kinase, and it is herein identified for the first time as having inositol 1,3,4,5,6-pentakisphosphate 1-phosphatase activity. These designations may be used interchangeably herein, along with the term “the enzyme.”

As used herein, “reducing” means a reduction that is statistically significant. The reduction can be in the amount or activity of the enzyme or the amount of mucous or one or more individual mucous components.

As used herein, “increasing” means an increase that is statistically significant. The increase can be in the amount or activity of the enzyme or the amount of mucous or one or more individual mucous components.

A method of treating a disease that is exacerbated by mucous secretion is provided, comprising the step of increasing the activity of inositol 1,3,4,5,6pentakisphosphate 1-phosphatase in a subject having a disease that is exacerbated by mucous, whereby mucous secretion is reduced and the disease is treated.

Diseases that are exacerbated by salt, fluid or mucous secretion include, but are not limited to, asthma, bronchitis and the common cold (e.g., rhinovirus infection and infection by other viruses that produce similar symptoms). The art recognizes that chloride secretion drives mucous secretion. See, for example, Zhou et al., (2001) Am. J. Respir. Cell Mol. Biol. 25: 486-491 and Nakanishi et al., (2001) Proc. Nat. Acad. Sci. USA 98: 5175-5180. These articles are herein incorporated by reference in their entirety, and specifically for their teaching regarding chloride secretion and salt, fluid and mucous secretion and their roles in asthma, bronchitis and the common cold. As the impact of mucous secretion on the condition of a subject is a matter or routine determination, any other conditions that are exacerbated by salt, fluid or mucous secretion are well within the scope of the present invention.

Treatment of a disease or condition as contemplated by the present invention can include any alleviation of any of the art-recognized symptoms of the disease or condition. The determination that treatment has occurred is a matter of routine practice by the skilled clinician, involving either measurement of salt, fluid or mucous levels or subjective reporting of the reduction of symptom severity by the patient. These parameters can be compared to pre-treatment levels in the same patient or they can be compared to objective standards based on what are art-recognized as normal levels.

The methods of the invention that involve a step of increasing the activity of inositol 1,3,4,5,6-pentakisphosphate 1-phosphatase can comprise the step of over-expressing inositol 1,3,4,5,6-pentakisphosphate 1-phosphatase.

Any compound that activates inositol 1,3,4,5,6-pentakisphosphate 1-phosphatase can be used in the present methods.

Genetic constructs can be used to over-express inositol 1,3,4,5,6 pentakisphosphate 1-phosphatase, thereby increasing the level of activity present. For example the coding sequence for inositol 1,3,4,5,6-pentakisphosphate 1-phosphatase can be inserted in an expression construct to express levels of the enzyme that exceed existing expression levels in the subject. A nucleic acid coding sequence (SEQ ID NO:1) for inositol 1,3,4,5,6-pentakisphosphate 1-phosphatase (SEQ ID NO:2) can be found in GenBank at accession number AF279372. The accession header for the sequence refers to the enzyme by its older nomenclature of “inositol 3,4,5,6 tetrakisphosphate 1-kinase/inositol 1,3,4-trisphosphate 5/6-kinase.” Other nucleic acid coding sequences that encode the enzyme described in SEQ ID NO:2 are, of course, also contemplated and would be recognized as such by those of skill in the art. Such nucleic acid coding sequences can include nucleic acid sequence that do not encode amino acid sequence and/or can include changes in the coding sequence that do not affect the encoded sequence (i.e., the codons encode the same amino acids due to the degeneracy of the amino acid code). Alternatively, genetic constructs can be used to over-express variants, mutants or fragments of inositol 1, 3, 4, 5, 6 pentakisphosphate 1-phosphatase that exhibit the desired phosphatase activity. Variants or mutants can be naturally-occurring variants or mutants or can be variants or mutants that are generated through random or directed mutagenesis. Mutagenesis, if used to generate variants or mutants, can be combinatorial in nature. Mutants or variants of inositol 1, 3, 4, 5, 6 pentakisphosphate 1-phosphatase can be those that contain conservative or non-conservative amino acid substitutions.

Conservative amino acid substitutions are well-known in the art, and include substitutions made on the basis of similarity in polarity, charge, solubility, hydrophobicity, hydrophilicity, amphipathicity and other factors. It is further recognized by those of skill in the art that substitutions, additions or deletions of a small percentage of amino acids (typically less than 7, 6, 5, or 4%, more typically less than 1%) in an encoded sequence are conservatively modified variations where the alterations result in the substitution of an amino acid with a chemically similar amino acid. Conservative substitution tables providing functionally similar amino acids are well known in the art. The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
   5) Isoleucine (I), Leucine (L), Methionine (M), Valine (V); and
6) Phenylalanine (F), Tyrosine (Y), Tryptophan (W).
   It will further be recognized that certain substitutions, additions and/or deletions can result from adoption of nucleic acid sequences that are advantageous for providing convenient cloning, restriction endonuclease and/or other features used by those of skill in the art. Further, there exist substitutions, additions and/or deletions which can be used to provide features useful in the purification of the polypeptide, including, but not limited to histidine tags and the like. Other substitutions, additions and/or deletions can be included to provide other properties useful for the safety, efficacy or effectiveness of the resulting protein or polypeptide.

A precursor of the enzyme can also be overexpressed to increase the synthesis of enzyme, as can a cellular signal that stimulates expression of the enzyme.

The genetic constructs that can be used to increase expression/activity of inositol 1,3,4,5,6-pentakisphosphate 1-phosphatase can be delivered using available vector technology as further described below. Since many of the conditions to be treated by the present method of reducing salt, fluid or mucous secretion involve the respiratory tract, delivery can be by inhalation as an aerosol (e.g., for asthma) or nasal spray (e.g., for cold symptoms) as further described below.

The methods of the invention that include a step of increasing the activity of inositol 1,3,4,5,6-pentakisphosphate 1-phosphatase can comprise administering to the subject a compound that activates the enzyme. Compounds that would activate the enzyme are shown herein to include alternate kinase substrates. Examples of alternate kinase substrates include, but are not limited to, inositol 1,2,4-trisphosphate, inositol 1,3,4-trisphosphate, inositol 3,4,6-trisphosphate, inositol 3,4,5-trisphosphate, inositol 3,5,6-trisphosphate and inositol 4,5,6-trisphosphate. The result of the present work shows these to be phosphorylated by recombinant inositol 1,3,4,5,6-pentakisphosphate 1-phosphatase.

The methods of the invention also can include the use of analogues of the alternate kinases to activate inositol 1,3,4,5,6-pentakisphosphate 1-phosphatase. Cell-permeant analogues in particular are known, and could be used. In fact, it is known in the art that cell-permeant inositol 1,3,4-trisphosphate elevates levels of inositol 3,4,5,6-trisphosphate in cells; i.e., inositol 1,3,4-trisphosphate acts in vivo as a specific regulator of cellular signaling by inositol 3,4,5,6-tetrakisphosphate. See Yang X, Rudolf M, Carew M A, Yoshida M, Nerreter V, Riley A M, Chung S K, Bruzik B S, Potter B V, Schultz C, Shears S B. J Biol Chem 1999 Jul. 2; 274(27):18973-80, which is incorporated herein by reference in its entirety and specifically for its teaching of the production of inositol polyphosphate analogues. At the time, it was thought that inositol 1,3,4-trisphosphate was inhibiting phosphorylation of inositol 3,4,5,6tetrakisphosphate by the 1-kinase. This may still be true, but the present data now shows something not previously known or suspected: inositol 1,3,4-trisphosphate activates inositol 1,3,4,5,6-pentakisphosphate 1-phosphatase. Accordingly, as will be recognized by those of skill in the art, inositol 1,3,4-triphosphate can be used to increase the amount of inositol 3,4,5,6 tetrakisphosphate by activation of the enzyme's 1-phosphatase activity.

The cell-permeant technology delivery system, patented by Inologic, can be useful in the practice of the invention. For examples of cell permeant compounds for use in the invention, see Li et al., 1997, “Membrane-Permeant Esters of Inositol Polyphosphates, Chemical Syntheses and Biological Applications”, Tetrahedron 53:12017-12040; Roemer et al., 1996, “Membrane-Permeant Analogues of the Putative Second Messenger myo-Inositol 3,4,5,6-Tetrakisphosphate”, J. Chem. Soc. Perkin Trans. 1:1683-1694; Rudolf et al., 1998, “A Membrane-Permeant, Bioactivatable Derivative of INS(1,3,4)P.sub.3 and Its Effect on Cl⁻ Secretion from T₈₄ Cells”, Bioorg. Med. Chem. 8:1857-1860; Schultz et al., 1998, “Membrane-Permeant, Bioactivatable Derivatives of Inositol Polyphosphates and Phosphoinositides”, Chapter XX in: Phosphoinositides: Chemistry, Biochemistry and Biomedical Applications, Bruzik, ed., Am. Chem. Soc. Symp. Ser. 718:232-243; U.S. Pat. No. 6,221,856, Inositol Derivatives for Inhibiting Superoxide Anion Production; U.S. Pat. No. 5,977,078, Inositol Polyphosphate Derivatives and Methods of Using Same; U.S. Pat. No. 5,880,099, Inositol Polyphosphates and Methods of Using Same, each of which is incorporated herein by reference in its entirety, and specifically for the teaching of cell permeant technology and cell permeant inositol polyphosphate substrate analogues.

For example, cell-permeant analogues of inositol 1,3,4-trisphosphate, inositol 3,4,6-trisphosphate, inositol 1,2,4-trisphosphate, inositol 3,4,5-trisphosphate, inositol 3,5,6-trisphosphate, and inositol 4,5,6-trisphosphate can include, but are not limited to, 2,5,6-tri-O-butyryl-myo-inositol 1,3,4-trisphosphate hexakis (acetoxymethyl) ester; 1,2,5-tri-O-butyryl-myo-inositol 3,4,6-trisphosphate hexakis (acetoxymethyl) ester; 1,2,4-tri-O-butyryl-myo-inositol 3,5,6-trisphosphate hexakis (acetoxymethyl) ester; 1,2,3-tri-O-butyryl-myo-inositol 4,5,6-trisphosphate hexakis (acetoxymethyl) ester; 1,2,6-tri-O-butyryl-myo-inositol 3,4,5-trisphosphate hexakis (acetoxymethyl) ester; and 3,5,6-tri-O-butyryl-myo-inositol 1,2,4-trisphosphate hexakis (acetoxymethyl) ester.

The synthesis of analogues of inositol polyphosphates is described in the literature. In addition to the references cited an incorporated by reference elsewhere in the application, the invention also includes the methods for the synthesis of cell-permeant inositol 1,3,4-trisphosphate described in “A membrane-permeant, bioactivatable derivative of Ins(1,3,4)P₃ and its effect on Cl(−)-secretion from T84 cells,” Rudolf M T, Traynor-Kaplan A E, Schultz C. Bioorg Med Chem Lett 1998 Jul. 21;8(14):1857-60, which is incorporated herein by reference in its entirety and specifically for its teaching of the production of inositol polyphosphate analogues. Given the teaching of this reference and others available in the art, one skilled in the art could routinely make other cell-permeant trisphosphates.

Another type of compound that can activate inositol 1,3,4,5,6-pentakisphosphate 1-phosphatase is a compound able to accept a phosphate group from the enzyme-phosphate intermediate. Without being limited to specific mechanisms, this could be what inositol 1,3,4- and 3,4,6-trisphosphate do when they are phosphorylated. Thus, generic phosphate acceptors are useful as activators of the enzyme.

The invention further provides a method of increasing salt or fluid secretion in a subject, comprising decreasing the activity of inositol 1,3,4,5,6-pentakisphosphate 1-phosphatase in the subject. The currently available or later-developed methods and compounds for decreasing the activity of this enzyme are contemplated to be within the scope of the invention. Examples of such compounds and methods are described herein and in other art that is incorporated by reference herein.

Having shown that an increase in secretion of salt and fluid results from decreased enzyme activity, a method of treating a disease that is treated by increased salt and fluid secretion is provided, comprising decreasing the activity of inositol 1,3,4,5,6-pentakisphosphate 1-phosphatase in a subject having a disease that is treated by increased salt and fluid secretion, whereby salt and fluid secretion is increased and the disease is treated.

An example of a disease that can be treated by increasing salt and fluid secretion is cystic fibrosis. The art recognizes that chloride secretion drives mucous secretion. See, for example, Ho MWY, Carew M A, Yang X, Shears S B: Regulation of chloride channel conductance by Ins(3,4,5,6)P₄; a phosphoinositide-initiated signalling pathway that acts downstream of Ins(1,4,5)P₃. In Frontiers in Molecular Biology: Biology of Phosphoinositides. Edited by Cockroft S. Oxford: Oxford University Press; 2000:298-319. This article is herein incorporated by reference in its entirety, and specifically for its teaching regarding chloride secretion, salt and fluid secretion and their roles in cystic fibrosis.

Treatment of a disease or condition by increasing salt and fluid secretion as contemplated by the present invention can include any alleviation of any of the art-recognized symptoms of the disease or condition. The determination that treatment has occurred is a matter of routine practice by the skilled clinician, involving either measurement of salt or fluid levels or subjective reporting of symptom severity by the patient. These parameters can be compared to pre-treatment levels in the same patient or they can be compared to objective standards based on what are art-recognized as normal levels.

A method of treating a disease that is characterized by improper insulin secretion or by improper nerve impulse transmission in neurons that relies on modulating the activity of Ins(3,4,5,6)P₄-regulated chloride channels in intracellular vesicles is also provided. For example, a treatment for a disease characterized by improper, inadequate secretion of insulin, such as type 2 diabetes, is provided, comprising the step of decreasing the activity of inositol 1,3,4,5,6-pentakisphosphate 1-phosphatase in a subject, thereby reducing the levels of Ins(3,4,5,6)P₄, and also thereby reducing down-regulation of insulin secretion associated with type 2 diabetes such that the disease is treated.

In the methods of the invention that include a step of decreasing the activity of inositol 1,3,4,5,6-pentakisphosphate 1-phosphatase, the reduction can comprise reducing the expression of inositol 1,3,4,5,6-pentakisphosphate 1-phosphatase. Genetic constructs can be used to reduce the expression of inositol 1,3,4,5,6 pentakisphosphate 1-phosphatase, thereby decreasing its activity. For example, there are several constructs that can be used to decrease expression of inositol 1,3,4,5,6-pentakisphosphate 1-phosphatase. For example antisense constructs are included, which comprise a sequence that interferes with the mRNA encoding the enzyme, and can cause degradation of the mRNA before it can be translated.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (K_{d})less than 10⁻⁶. It is more preferred that antisense molecules bind with a K_{d} less than 10⁻⁸. It is also more preferred that the antisense molecules bind the target molecule with a K_{d} less than 10⁻¹⁰. It is also preferred that the antisense molecules bind the target molecule with a K_{d} less than 10⁻¹². A representative sample of methods and techniques which aid in the design and use of antisense molecules can be found in the following non-limiting list of U.S. Pat. Nos.: 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Additionally, double-stranded RNA constructs have been used in plants and drosophila as a specific gene silencer. See, for example, Clemens, J. C., Worby, C. A., Simonson-Leff, N., Muda, M., Maehama, T., Hemmings, B. A., and Dixon, J. E. (2000) Proc. Nat. Acad. Sci. USA 12, 6499-6503, which is herein incorporated by reference in its entirety, and specifically for its teaching regarding double-stranded RNA constructs and their use as inhibitor of expression.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹² M. It is preferred that the aptamers bind the target molecule with a K_{d} less than 10⁻⁶. It is more preferred that the aptamers bind the target molecule with a K_{d} less than 10⁻⁸. It is also more preferred that the aptamers bind the target molecule with a K_{d} less than 10⁻¹⁰. It is also preferred that the aptamers bind the target molecule with a K_{d} less than 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a K_{d} with the target molecule at least 10 fold lower than the K_{d} with a background binding molecule. It is more preferred that the aptamer have a K_{d} with the target molecule at least 100 fold lower than the K_{d} with a background binding molecule. It is more preferred that the aptamer have a K_{d} with the target molecule at least 1000 fold lower than the K_{d} with a background binding molecule. It is preferred that the aptamer have a K_{d} with the target molecule at least 10000 fold lower than the K_{d} with a background binding molecule. It is preferred when doing the comparison for a polypeptide for example, that the background molecule be a different polypeptide. For example, when determining the specificity of aptamers, the background protein could be actin. Representative examples of how to make and use aptamers to bind a variety of different target molecules can be found in the following non-limiting list of U.S. Pat. Nos.: 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos.: 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos.: 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos.: 5,595,873 and 5,652,107). There are also a number of ribozymes that are not found in natural systems, but which have been engineered to catalyze specific reactions de novo (for example, but not limited to the following U.S. Pat. Nos.: 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos.: 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a K_{d} less than 10⁻⁶. It is more preferred that the triplex forming molecules bind with a K_{d} less than 10^{31 8}. It is also more preferred that the triplex forming molecules bind the target molecule with a K_{d} less than 10⁻¹⁰. It is also preferred that the triplex forming molecules bind the target molecule with a K_{d} less than 10⁻¹². Representative examples of how to make and use triplex forming molecules to bind a variety of different target molecules can be found in the following non-limiting list of U.S. Pat. Nos.: 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

In the methods of the invention including a step of decreasing the activity of inositol 1,3,4,5,6-pentakisphosphate 1-phosphatase, the step can comprise adiministering to the subject a compound that decreases the activity of inositol 1,3,4,5,6-pentakisphosphate 1-phosphatase. Examples of compounds that can decrease the activity of inositol 1,3,4,5,6 pentakisphosphate 1-phosphatase include analogues or other mimics of inositol 1,3,4,5,6-pentakisphosphate that successfully compete with and/or inhibit inositol 1,3,4,5,6-pentakisphosphate dephosphorylation. Analogues of inositol 1,3,4-trisphosphate and inositol 3,4,6-trisphosphate that bind to the enzyme but are not phosphorylated should also inhibit 1-phosphatase.

Examples of inositol phosphates structurally similar to inositol 1,3,4,5,6-pentakisphosphate include, but are not limited to inositol 1,2,4,5,6 pentakisphosphate, inositol 2,3,4,5,6 pentakisphosphate, inositol 1,2,3,4,5 pentakisphosphate, inositol 1,2,3,4,6 pentakisphosphate, inositol 1,2,3,5,6 pentakisphosphate. These can be used to decrease activity of the enzyme by acting as competitors for the enzyme's normal substrate. Methods for synthesizing these compounds are known in the art. For example, see Chung S K, Chang Y T, Lee E J, Shin B G, Kwon Y U, Kim K C, Lee D H, Kim M J, “Syntheses of two enantiomeric pairs of myo-inositol(1,2,4,5,6) and −(1,2,3,4,5) pentakisphosphate,” Bioorg Med Chem Lett 1998 Jun. 16;8(12):1503-6, which is incorporated herein by reference in its entirety and specifically for its teaching of the production of inositol polyphosphates structurally similar to inositol 1,3,4,5,6-pentakisphosphate.

The invention provides a method of decreasing chloride secretion from calcium-activated chloride channels in a cell, comprising decreasing inositol 3,4,5,6 phosphate kinase activity and/or increasing inositol 1,3,4,5,6-pentakisphosphate 1-phosphatase activity in the cell.

Also provided is a method of increasing chloride secretion from calcium-activated chloride channels in a cell, comprising increasing inositol 3,4,5,6 phosphate kinase activity and/or decreasing inositol 1,3,4,5,6-pentakisphosphate 1-phosphatase activity in the cell.

The invention provides a method of decreasing chloride secretion from calcium-activated chloride channels in a cell, comprising increasing inositol 3,4,5,6 tetrakisphosphate in the cell by decreasing inositol 3,4,5,6 phosphate kinase activity and/or increasing inositol 1,3,4,5,6-pentakisphosphate 1-phosphatase activity in the cell.

Also provided is a method of increasing chloride secretion from calcium-activated chloride channels in a cell, comprising deceasing inositol 3,4,5,6 tetrakisphosphate in the cell by increasing inositol 3,4,5,6 phosphate kinase activity and/or decreasing inositol 1,3,4,5,6-pentakisphosphate 1-phosphatase activity in the cell.

In the methods of the invention, the cell can be an epithelial cell, a neuron or a smooth muscle cell. Chloride channel conductance in these cell types is known to be regulated by inositol 3,4,5,6-tetrakisphosphate. The methods of the invention can increase or decrease chloride secretion from calcium-activated chloride channels in one or more of these cell types at the same or approximately the same time or in other temporal relationships.

Also provided by the invention is a method of preventing or reducing inositol 3,4,5,6 tetrakisphosphate 1-phosphate-mediated inhibition of insulin release, comprising decreasing the activity of inositol 1,3,4,5,6 pentakisphosphate 1-phosphatase in the subject. Decreasing the amount of Ins(3,4,5,6)P₄ can also be accomplished by increasing the activity of inositol 1,3,4,5,6 pentakisphosphate 1-phosphatase in the subject under certain conditions as will be recognized by those of skill in the art. Specifically, as the enzyme catalyzes both the phosphorylation and dephosphorylation, under conditions that favor formation of 1,3,4,5,6 pentakisphosphate from 3,4,5,6 tetrakisphosphate, increasing the activity of the enzyme will help to deplete (i.e., reduce) the level of 3,4,5,6 tetrakisphosphate. Thus, as one of skill in the art will recognize, under certain specific, albeit not necessarily normal physiological conditions, the increasing the activity of the enzyme can have the same effect as reducing the activity of the enzyme under normal physiological conditions and vice versa.

Also provided by the invention is a method of treating a disease that is exacerbated by inhibition of insulin secretion, comprising decreasing the activity of inositol 1,3,4,5,6 pentakisphosphate 1-phosphatase in a subject having a disease that is exacerbated by inhibition of insulin secretion, whereby the inhibition of insulin secretion is reduced and the disease is treated. It is contemplated that the disease to be treated by use of the methods of the invention is type 2 diabetes.

Reduction of the activity of inositol 1,3,4,5,6 pentakisphosphate 1-phosphatase can include reducing expression of inositol 1,3,4,5,6 pentakisphosphate 1-phosphatase or can include decreasing the activity of inositol 1,3,4,5,6 pentakisphosphate 1-phosphatase of a combination of both. If the method includes reducing the activity of enzyme present, the method can include administering to the subject a compound that inhibits inositol 1,3,4,5,6 pentakisphosphate 1-phosphatase.

The invention also includes a method of increasing inositol 3,4,5,6 tetrakisphosphate 1-phosphate-mediated inhibition of insulin release, comprising increasing the activity of inositol 1,3,4,5,6 pentakisphosphate 1-phosphatase in the subject and methods of treating a disease that is treated by increasing levels of inositol 3,4,5,6 tetrakisphosphate thereby modulating inositol 3,4,5,6 tetrakisphosphate 1-phosphate-regulated chloride channels in intracellular vesicles. Such method can comprise altering the activity of inositol 1,3,4,5,6 pentakisphosphate 1-phosphatase in a subject having a disease that is treated by decreased inositol 1,3,4,5,6 pentakisphosphate 1-phosphatase activity, whereby the disease is treated or can include altering the activity of 1,3,4,5,6 pentakisphosphate 1-phosphatase in a subject having a disease that is treated by increased inositol 1,3,4,5,6 pentakisphosphate 1-phosphatase activity

The invention also includes a method of treating a disease that is treated by decreasing levels of inositol 3,4,5,6 tetrakisphosphate thereby modulating inositol 3,4,5,6 tetrakisphosphate 1-phosphate-regulated chloride channels in intracellular vesicles, comprising altering the activity of inositol 1,3,4,5,6 pentakisphosphate 1-phosphatase in a subject having a disease that is treated by increased inositol 1,3,4,5,6 pentakisphosphate 1-phosphatase activity, whereby the disease is treated. In methods of the present invention wherein the activity is modulated, the modulation can include altering the expression of inositol 1,3,4,5,6 pentakisphosphate 1-phosphatase. Alteration of enzyme expression can transient or permanent in nature. Alteration of enzyme expression can be global or localized in nature. If localized, it is contemplated that the altered expression be limited to or most significantly effected in specific organs, tissues, cells, organelles or vesicles. It is contemplated that modulating the activity of inositol 1,3,4,5,6 pentakisphosphate 1-phosphatase can comprise administering to the subject a compound that increases or decreases the activity of inositol 1,3,4,5,6 pentakisphosphate 1-phosphatase.

The present invention also provides a method of decreasing chloride secretion from calcium-activated chloride channels in an intracellular vesicle, comprising decreasing inositol 3,4,5,6 phosphate kinase activity in the cell.

The present invention also provides a method of increasing chloride secretion from calcium-activated chloride channels in an intracellular vesicle, comprising increasing inositol 3,4,5,6 phosphate kinase activity in the intracellular vesicle.

The present invention also provides a method of increasing the activity of calcium-activated chloride channels in an intracellular vesicle, comprising decreasing inositol 1,3,4,5,6 pentakisphosphate 1-phosphatase activity in the intracellular vesicle.

The invention provides methods of identifying compounds that can increase the activity of inositol 1,3,4,5,6 pentakisphosphate 1-phosphatase, comprising contacting a system (in vivo or in vitro) that expresses inositol 1,3,4,5,6 pentakisphosphate 1-phosphatase activity with the compound, and detecting an increase in the activity of inositol 1,3,4,5,6 pentakisphosphate 1-phosphatase. Any class of compounds can be tested in the present system to identify nucleic acids, proteins, small molecules, etc. that increase enzyme activity. Representative examples of systems that express enzyme activity that can be used to identify modulators (activators) of enzyme activity are described in the examples. An increase in activity indicates that the compound is an activator of the enzyme. The increase in activity can by detected by detecting an increase in 3,4,5,6-tetrakisphosphate using methods described herein. The increase in activity can be detected by detecting a decrease in the activity of calcium activated chloride channels using methods described herein. The increase can be detected by detecting a decrease in salt, fluid or mucous secretion, for example in airway epithelial cells using methods described herein. The air-liquid interface cultures described herein are well suited to such measurement because there is no medium on the apical surface of the cells; thus any fluid that accumulates there can be easily aspirated and analyzed for amount and content. The increase can be detected by detecting a decrease in salt, fluid or mucous secretion in airway epithelium using methods described herein and those well-known in the art. For example, bronchoscopy and aspiration of fluid from the bronchii or trachea can be used to measure salt, fluid or mucous levels in the lungs of a subject. See, for example Howarth P H. “Why particle size should affect clinical response to inhaled therapy,” J Aerosol Med. 2001;14 Suppl 1:S27-34, Review; Peros-Golubicic T, Ivicevic A, Bekic A, Alilovic M, Tekavec-Trkanjec J, Smojver-Jezek S. “Lung lavage neutrophils, neutrophil elastase and albumin in the prognosis of pulmonary sarcoidosis,” Coll Antropol. 2001 June;25(1):349-55; and Patelli M, Agli L L, Poletti V, Trisolini R, Cancellieri A, Lacava N, Falcone F, Boaron M. “Role of fiberscopic transbronchial needle aspiration in the staging of N2 disease due to non-small cell lung cancer,” Ann Thorac Surg. 2002 February; 73(2):407-11, which are incorporated herein by reference in their entireties, and specifically for their teaching regarding obtaining and analyzing tracheal and lung fluids. Similarly, levels of salt, fluid or mucous in the nose can be measured by intranasal swabbing and analysis of the fluid obtained. Alteration of activity can be detected by monitoring the alteration in the amount of insulin secreted from cells, or it can be detected from physiological effects resulting from the secretion of insulin including, but not limited to, measurement of blood sugar levels. Alternatively, alteration of activity or other physiological effects related to, but not limited to the secretion of insulin, can be monitored by the observation of processes related to the physiological process to be affected, for example, those process related to the secretion of insulin. In the case of insulin secretion, these related processes include, but are not limited to, acidification of the vesicular interior of insulin granules in pancreatic β-cells, fusion of insulin granules with the plasma membrane. Monitoring these processes can include visualization of the structures and processes or can include monitoring of electrochemical properties recognized by those of skill in the art to be relevant and useful such as conductance or capacitance measurements.

The invention provides methods of identifying compounds that can decrease the activity of inositol 1,3,4,5,6 pentakisphosphate 1-phosphatase, comprising contacting a system (in vivo or in vitro) that expresses inositol 1,3,4,5,6 pentakisphosphate 1-phosphatase activity with the compound, and detecting a decrease in the activity of inositol 1,3,4,5,6 pentakisphosphate 1-phosphatase. Representative examples of systems that express enzyme activity that can be used to identify modulators (inhibitors) of enzyme activity are described in the examples. Any class of compounds can be tested in the present system to identify nucleic acids, proteins (e.g., antibodies), small molecules, etc. that decrease enzyme activity. A decrease in activity indicates that the compound is and inhibitor of enzyme activity. The decrease in activity can by detected by detecting a decrease in 3,4,5,6-tetrakisphosphate using methods described herein. The decrease in activity can be detected by detecting a increase in the activity of calcium activated chloride channels using methods described herein. The decrease can be detected by detecting an increase in salt, fluid or mucous secretion, for example in airway epithelial cells and in airway epithelium using methods described herein and known in the art. Alternatively, alteration of activity can be detected by monitoring the alteration in the amount of insulin secreted from cells, or it can be detected from physiological effects resulting from the secretion of insulin. Alternatively, alteration of activity or other physiological effects related to, but not limited to the secretion of insulin, can be monitored by the observation of processes related to the secretion of insulin. These related processes include, but are not limited to, acidification of the vesicular interior of insulin granules in pancreatic β-cells, fusion of insulin granules with the plasma membrane. Monitoring these processes can include visualization of the structures and processes or can include monitoring of electrochemical properties recognized by those of skill in the art to be relevant and useful such as conductance or capacitance measurements.

Combinatorial chemistry can be used to prepare libraries of compounds that can increase or decrease the activity of inositol 1,3,4,5,6 pentakisphosphate 1-phosphatase.

The disclosed compositions and relationships can be used as targets for any combinatorial technique to identify molecules or macromolecular molecules that interact with the enzyme in a desired way. Also disclosed are the compositions that are identified through combinatorial techniques or screening techniques in the enzyme is used as the target in a combinatorial or screening protocol.

It is understood that when using the disclosed compositions in combinatorial techniques or screening methods, molecules, such as macromolecular molecules, will be identified that have particular desired properties such as inhibition or stimulation or the target enzyme's function. The molecules identified and isolated when using the disclosed compositions are also disclosed as part of the invention. Thus, the products produced using the combinatorial or screening approaches that involve the disclosed compositions are also considered herein disclosed.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332, which are herein incorporated by reference at least for their material related to phage display and methods relate to combinatorial chemistry).

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997), which is incorporated by in its entirety).

Another preferred method for combinatorial methods designed to isolate peptides is described in Cohen et al. (Cohen B. A., et al., Proc. Natl. Acad. Sci. USA 95(24): 14272-7 (1998), which is incorporated by reference at least for the material related to combinatorial chemistry). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636, which are incorporated by reference at least for their material related to combinatorial chemistry.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. No. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofirans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (United States patent 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

As used herein combinatorial methods and libraries include traditional screening methods and libraries as well as methods and libraries used in iterative processes.

Computer assisted drug design can be used to identify compounds that increase or decrease the activity of inositol 1,3,4,5,6 pentakisphosphate 1-phosphatase. The disclosed compositions can be used as targets for any molecular modeling technique to identify either the structure of the disclosed compositions or to identify potential or actual molecules, such as small molecules, which interact in a desired way with the disclosed compositions.

It is understood that when using the disclosed compositions in modeling techniques, molecules, such as macromolecular molecules, will be identified that have particular desired properties such as inhibition or stimulation or the target enzyme's function. The molecules identified and isolated when using the disclosed methods and compositions are also disclosed. Thus, the products produced using the combinatorial or screening approaches that involve the disclosed methods and compositions are also considered herein disclosed.

Thus, one way to isolate molecules that interact with the enzyme is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

Protocols for experimental or clinical trials of drugs, including for example, peptides and small molecules, in therapy of different disease will vary in accordance with the disease to be treated and with the agent used in the treatment of the disease. For example, for therapy of mucous diseases of the lungs (e.g., bronchitis, cystic fibrosis, allergic asthma, etc.), agents can be delivered by inhalation of aerosols or inhalation of biodegradable materials, including, but not limited to, polymeric materials which contain the agents/drugs/peptides (Edwards et al., Science 276: 1868-1871 (1997)). Therapies or trials for therapies of nasal diseases can also utilize aerosols.

Specific targeting of organs or tissues is also contemplated. Such organs that might be targeted include, but are not limited to, the pancreas (for treatment of diabetes and diabetes-type disorders). Consequently, the methods of treatment and compositions for such treatment provided by the present invention include proteins or other drugs that can be introduced into the bloodstream that contain ligands design to specifically target organs of interest. The proteins or drugs of the invention can be delivered into the bloodstream in vesicles. For example, pH sensitive membrane permease enzymes can be included within the vesicles used to deliver the drugs so that once the vesicle is absorbed into the cell, the drugs or peptides that are inside the drug-delivery vesicle can be delivered to the cell (see, for example, Stayton et al. Bioconjug. Chein 13: 996-1001 (2002)).

Compositions comprising compounds that increase or decrease the activity of inositol 1,3,4,5,6 pentakisphosphate 1-phosphatase are provided. Such compositions can be made by combining a compound identified in one of the methods described above with a pharmaceutically acceptable carrier.

The invention provides therapeutic methods and therapeutic compositions. The compositions can also be administered in vivo in a pharmaceutically acceptable carrier. By “pharmaceutically acceptable” is meant a material that is not biologically or otherwise undesirable, i.e., the material may be administered to a subject, along with the composition, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including intranasally, ophthalmically, vaginally, rectally), orally, by inhalation (including aerosol), or parenterally, for example by intravenous drip, subcutaneous, intraperitoneal or intramuscular injection. The disclosed compositions and combinations and mixtures can be administered intravenously, intraperitoneally, intramuscularly, subcutaneously, intracavity, or transdermally, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the composition. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the disorder being treated, the particular composition used, its mode of administration and the like. Thus, it is not possible to specify an exact amount for every composition. However, an appropriate amount can be determined by one of ordinary skill in the art using only routine experimentation given the teachings herein.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable. Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any contraindications. Dosage can vary, and can be administered in one or more dose administrations daily, for one or several days.

Both nucleic acid and non-nucleic acid activators and inhibitors of the activity of inositol 1,3,4,5,6 pentakisphosphate 1-phosphatase can be delivered to cells either in vitro or in vivo. There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352,815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)).

As used herein, plasmid or viral vectors are agents that transport the disclosed nucleic acids into the cell without degradation and include a promoter yielding expression of the gene in the cells into which it is delivered. In some embodiments the delivery molecules are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families, which share the properties of these viruses, which make them suitable for use as vectors. Retroviruses include Murine Maloney Leukemia virus, MMLV, and retroviruses that express the desirable properties of MMLV as a vector. Because retroviral vectors are able to carry a larger genetic payload, i.e., a transgene or marker gene, than other viral vectors, they are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector, which has been engineered so as to suppress the immune response of the host organism, elicited by the viral antigens. Preferred vectors of this type will carry coding regions for Ins(1,3,4,5,6)P₅ 1-phosphatase or variant or mutants thereof.

Viral vectors can have higher transaction (ability to introduce genes) abilities than chemical or physical methods to introduce genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promotor cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

A retrovirus is an animal virus belonging to the virus family of Retroviridae, including any types, subfamilies, genus, or trophisms. Retroviral vectors, in general, are described by Verma, I. M., Retroviral vectors for gene transfer. In Microbiology-1985, American Society for Microbiology, pp. 229-232, Washington, (1985), which is incorporated by reference herein. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules, which are needed in cis, for the replication, and packaging of the replicated virus. Typically a retroviral genome, contains the gag, pol, and env genes which are involved in the making of the protein coat. It is the gag, pol, and env genes, which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serve as the priming site for the synthesis of the second strand of DNA synthesis, and specific sequences near the ends of the LTRs that enable the insertion of the DNA state of the retrovirus to insert into the host genome. The removal of the gag, pol, and env genes allows for about 8 kb of foreign sequence to be inserted into the viral genome, become reverse transcribed, and upon replication be packaged into a new retroviral particle. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery, but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell, but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)). Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol. 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. In another preferred embodiment both the E1 and E3 genes are removed from the adenovirus genome.

Another type of viral vector is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P_{4.1} C vector produced by Avigen, San Francisco, Calif., which can contain the herpes simplex virus thymidine kinase gene, HSV-tk, and/or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus.

Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference for material related to the AAV vector.

The vectors of the present invention thus provide DNA molecules, which are capable of integration into a mammalian chromosome without substantial toxicity.

The inserted genes in viral and retroviral usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Molecular genetic experiments with large human herpesviruses have provided a means whereby large heterologous DNA fragments can be cloned, propagated and established in cells permissive for infection with herpesviruses (Sun et al., Nature genetics 8: 33-41, 1994; Cotter and Robertson,. Curr Opin Mol Ther 5: 633-644, 1999). These large DNA viruses (herpes simplex virus (HSV) and Epstein-Barr virus (EBV), have the potential to deliver fragments of human heterologous DNA >150 kb to specific cells. EBV recombinants can maintain large pieces of DNA in the infected B-cells as episomal DNA. Individual clones carried human genomic inserts up to 330 kb appeared genetically stable The maintenance of these episomes requires a specific EBV nuclear protein, EBNA1, constitutively expressed during infection with EBV. Additionally, these vectors can be used for transfection, where large amounts of protein can be generated transiently in vitro. Herpesvirus amplicon systems are also being used to package pieces of DNA >220 kb and to infect cells that can stably maintain DNA as episomes.

Other useful systems include, for example, replicating and host-restricted non-replicating vaccinia virus vectors.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### Example 1

Regulation of Ins(3456)P₄ Signalling by a Reversible Kinase/Phosphatase.

Enzyme Assays

Inositol phosphate phosphatase activity was assayed in buffer containing 100 mM KCl, 20 mM HEPES pH 7.2, 5 mM ADP, 6 mM MgSO₄, 0.3 mg/ml bovine serum albumin. Assays were acid-quenched, neutralized and analyzed by HPLC using a Synchropak Q100 columm (Caffrey et al., “Expanding Coincident Signaling by PTEN through its Inositol 1,3,4,5,6-Pentakisphosphate 3-phosphatase Activity,” FEBS Lett 499: 6-10 (2001)); 1 ml fractions were collected for 70 min., followed by 0.5 ml fractions. Inositol phosphate kinase activity was assayed in buffer containing 100 mM KCl, 20 mM HEPES pH 7.2, 5 mM ATP, 10 mM phosphocreatine, 6 mM MgSO₄, 10 μg/ml creatine phosphokinase (Calbiochem), 0.3 mg/ml bovine serum albumin. Some assays were acid-quenched and analyzed by HPLC using a Synchropak Q100 column. Other reactions were heat-inactivated (95° C., 3 min) and analyzed by a metal dye detection, HPLC method (Mayr, “A novel metal-dye detection sysytem permits picomolar-range h.p.l.c. analysis of inositol polyphosphates from non-radioactively labelled cell or tissue specimens,” Biochem J 254: 585-591 (1988); Adelt et al., “Enzyme-assisted total synthesis of the optical antipodes D-myo-inositol 3,4,5-trisphosphate and D-myo-inositol 1,5,6-trisphosphate: aspects of their structure-activity relationship to biologically active inositol phosphates,” J Med Chem 42: 1262-1273 (1999)).

Transfection of the 1-kinase/1-Phosphatase Gene into T₈₄ Cells

T₈₄ cells were cultured at 37° C. (5% CO₂: 95% air) in Iscove's Modified Dulbecco's medium (Hyclone) supplemented with 5% (w/v) FBS (Hyclone, Logan, UT), 100 U/ml penicillin, 0.1 mg/ml streptomycin and 0.8 mg/ml G418 selection agent. The gene previously denoted a Ins(3,4,5,6)P₄ 1-kinase [4] was cloned into PCMV-Tag4 expression vector between the BamHI and xhoI sites after the stop codon was removed. The transfection was carried out in a 60 mm dish using LipoTAXI Mammalian Transfection Kit from Stratagene (La Jolla, Calif.), according to the manufacturer's instructions. Control transfections were performed at the same time using PCMV-Tag4 vector alone. Data shown are from one pair of cell-lines stably transfected with either enzyme or vector; similar data were obtained with two additional, independently and stably transfected pairs of cell-lines.

Electrophysiology

T₈₄ cells were detached by incubation with 0.25% (w/v) trypsin/0.02% (w/v) EDTA for 5-6 min. After trypsin removal, 200 μl aliquots (1.5×10⁶/ml) were seeded into the circular wells (area=0.45 cm²) of permeable supports, made by gluing (Silastic® sealant, Dow Corning, Midland, Mich.) a Sylgard® ring to filters composed of mixed cellulose esters (Millipore Corp., Bedford, Mass.). Seeded supports were floated on culture medium and incubated for 11-14 days before being mounted in modified Snapwell holders in Using chambers (EasyMount System, Physiologics Instruments, San Diego, Calif.). Transcellular Cl⁻ flux was then recorded as the short-circuit current as previously described [1]. The resistance of the monolayers were (vector) 346±44 ohms/cm² (mean±se, n=12) and (kinase) 354±62 ohms/cm² (mean±se, n=12). Whole-cell Cl⁻ currents in CFPAC—I cells were measured at +40 mV as previously described using an intracellular Ca²⁺-BAPTA buffer to clamp free [Ca²⁺] to 0.5 μM [8].

Phosphorylation of Alternative Kinase Substrates

Activity of 3.4 ug of inositol 3,4,5,6-tetrakisphosphate 1-kinase was assayed for 4 hr. at 37° C. in 200 μl buffer containing 100 mM KCl, 20 mM HEPES pH 7.2, 5 mM ATP, 10 mM phosphocreatine, 6 mM MgSO₄, 10 μg/ml creatine phosphokinase (Calbiochem), 0.3 mg/ml bovine serum albumin and 50 uM of either inositol 3,4,5-trisphosphate, 4,5,6-trisphosphate or inositol 3,5,6-trisphosphate. Reactions were heat-inactivated (95° C., 3 min) and analyzed by a metal dye detection, HPLC method [19,20]. By using appropriate standard materials, it was established that inositol 3,4,5-trisphosphate was phosphorylated to inositol 3,4,5,6-tetrakisphosphate, inositol 4,5,6trisphosphate was phosphorylated to inositol 1,4,5,6-tetrakisphosphate and that inositol 3,5,6-trisphosphate was phosphorylated to inositol 1,3,5,6-tetrakisphosphate.

Materials

American Type Cell Culture (Manassas, Va.) supplied the CFPAC-1 cells and T₈₄ cells. Recombinant human Ins(3,4,5,6)P₄ 1-kinase was expressed in E. coli and purified (Yang et al., “Multitasking in Signal Transduction by a Promiscuous Human Ins(3,4,5,6)P₄ 1-Kinase/Ins(1,3,4)P₃ 5/6-Kinase,” Biochem J 351: 551-555 (2000)). Non-radiolabeled Ins(1,3,4)P₃ and Ins(1,3,4,5,6)P₅ were purchased from CellSignals Inc (Lexington, Ky.). The details of the synthesis of non-radiolabeled Ins(3,4,5)P₃, Ins(3,5,6)P₃, Ins(4,5,6)P₃ and Ins(3,4,6)P₃ (as sodium salts) will be published separately by S.-K. C. Ins(3,4,6)P₃ was also prepared by a different synthetic route (Mills et al., “Synthesis of D- and L-myo-inositol 1,4,6-trisphosphate, regioisomers of a ubiquitous second messenger,” J Org Chem 61: 8980-8987 (1996)). [³H]Ins(1,3,4,5,6)P₅ was prepared as previously described (Zhang et al., “The Transcriptional Regulator, Arg82, is a Hybrid Kinase with both Monophosphoinositol- and Diphosphoinositol-Polyphosphate Synthase Activity,” FEBS Lett 494: 208-212 (2001)). [³H]Ins(1,3,4)P₃ was prepared from [³H]Ins(1,3,4,5)P₄ (New England Nuclear) using recombinant Ins(1,4,5)P₃/Ins(1,3,4,5)P₄ 5-phosphatase (Erneux et al., “Production of recombinant human brain type I inositol-1,4,5-trisphosphate 5-phosphatase in Escherichia coli. Lack of phosphorylation by protein kinase C,” Eur J Biochem 234: 598-602 (1995)). [³H]Ins(1,3,4)P₃ was converted to [³H]Ins(1,3,4,6)P₄ using Ins(1,3,4)P₃ 6-kinase activity (Yang et al., “Multitasking in Signal Transduction by a Promiscuous Human Ins(3,4,5,6)P₄ 1-Kinase/Ins(1,3,4)P₃ 5/6-Kinase,” Biochem J 351: 551-555 (2000)). [³H]Ins(3,4)P₂ was prepared by alkaline phosphatase attack on [³H]Ins(1,3,4)P₃ in 20 mM glycine (pH 9.0 with KOH). [³H]Ins(1,4)P₂ was purchased from New England Nuclear.

Results and Discussion

Signalling entities are frequently controlled by quite delicate shifts in the dynamic balance of regulatory signals with competing impacts. Ion channels provide particularly impressive examples of the degree of signal amplification that can result; switching the conductance state of a single channel can influence the transmembrane movement of millions of ions per second (Clapham, “How to lose your hippocampus by working on chloride channels,” Neuron 2001, 29: 1-6 (2001)). Both stimulatory (Ca²⁺ and CaMKII) and inhibitory (Ins(3,4,5,6)P₄) signals converge on the family of so-called “Ca²⁺-activated” Cl⁻ channels (Ho et al., “Regulation of chloride channel conductance by Ins(3,4,5,6)P₄; a phosphoinositide-initiated signalling pathway that acts downstream of Ins(1,4,5)P₃,” In Frontiers in Molecular Biology: Biology of Phosphoinositides. Edited by Cockroft S. Oxford: Oxford University Press; 298-319 (2000); Ho et al., “Regulation of a Human Chloride Channel: A Paradigm for Integrating Input from Calcium, CaMKII and Ins(3,4,5,6)P₄,” J Biol Chem 276: 18673-18680 (2001); xie et al., “Inositol 3,4,5,6-tetrakisphosphate inhibits the calmodulin-dependent protein kinase II-activated chloride conductance in T84 colonic epithelial cells,” J Biol Chem 271: 14092-14097 (1996)). Thus, receptor-dependent changes in Ins(3,4,5,6)P₄ levels (Menniti et al., “Origins of myo-inositol tetrakisphosphates in agonist-stimulated rat pancreatoma cells. Stimulation by bombesin of myo-inositol (1,3,4,5,6) pentakisphosphate breakdown to myo-inositol (3,4,5,6) tetrakisphosphate,” J Biol Chem 265: 11167-11176 (1990)) is a topic of general biological significance, in that it impacts upon regulation of salt and fluid secretion from epithelial cells (Carew et al., “Ins(3,4,5,6)P₄ inhibits an apical calcium-activated chloride conductance in polarized monolayers of a cystic fibrosis cell-line,” J Biol Chem 275: 26906-26913 (2000)), cell volume homeostasis (Nilius et al., “Inhibition by inositoltetrakisphosphates of calcium—and volume-activated Cl⁻ currents in macrovascular endothelial cells,” Pflügers Arch—Eur J Physiol 435: 637-644 (1998)), and electrical excitability in neurones and smooth muscle (Ho et al., “Regulation of chloride channel conductance by Ins(3,4,5,6)P₄; a phosphoinositide-initiated signalling pathway that acts downstream of Ins(1,4,5)P₃,” In Frontiers in Molecular Biology: Biology of Phosphoinositides. Edited by Cockroft S. Oxford: Oxford University Press; 298-319 (2000); Frings et al., “Neuronal Ca²⁺-activated Cl⁻ channels—homing in on an elusive channel species,” Prog Neurobiol 60: 247-289 (2000)). Unfortunately, understanding of the cellular control of Ins(3,4,5,6)P₄-signaling has been rudimentary, because the pathway of Ins(3,4,5,6)P₄ synthesis has not previously been characterized.

One possibility is that Ins(3,4,5,6)P₄ originates by phosphorylation of an InsP₃ precursor. We examined all potential InSP₃ precursors of Ins(3,4,5,6)P₄ by individually introducing them at 100 μM concentrations into human pancreatoma CFPAC-1 cells; the bio-assay for Ins(3,4,5,6)P₄ synthesis by any endogenous kinase was the sensitivity of calcium-activated Cl⁻ channels to Ins(3,4,5,6)P₄ (IC₅₀=3 μM) (Ho et al., “Regulation of chloride channel conductance by Ins(3,4,5,6)P₄; a phosphoinositide-initiated signalling pathway that acts downstream of Ins(1,4,5)P₃,” In Frontiers in Molecular Biology: Biology of Phosphoinositides. Edited by Cockroft S. Oxford: Oxford University Press; 298-319 (2000)); Ho et al., “Inositol 3,4,5,6-tetrakisphosphate specifically inhibits a receptor-mediated Ca²⁺-dependent Cl⁻ current in CFPAC-1 cells,” Am J Physiol 272: C1160-C1168 (1997)). Control whole-cell Cl⁻ current (54±3 pA/pF, n—17) was reduced to 24±3 pA/pF (n=7) by 10 μM Ins(3,4,5,6)P₄. In contrast, Ins(4,5,6)P₃ (56±7 pA/pF, n=10), Ins(3,4,6)P₃ (68±5 pA/pF, n=12), Ins(3,4,5)P₃ (61±7 pA/pF, n=9) and Ins(3,5,6)P₃ (60±6 pA/pF, n=13) showed no inhibitory effect. These results indicate that no substantial InsP₃ phosphorylation to Ins(3,4,5,6)P₄ occurred, while also demonstrating that all four phosphates of Ins(3,4,5,6)P₄ contribute to its exquisite specificity of action.

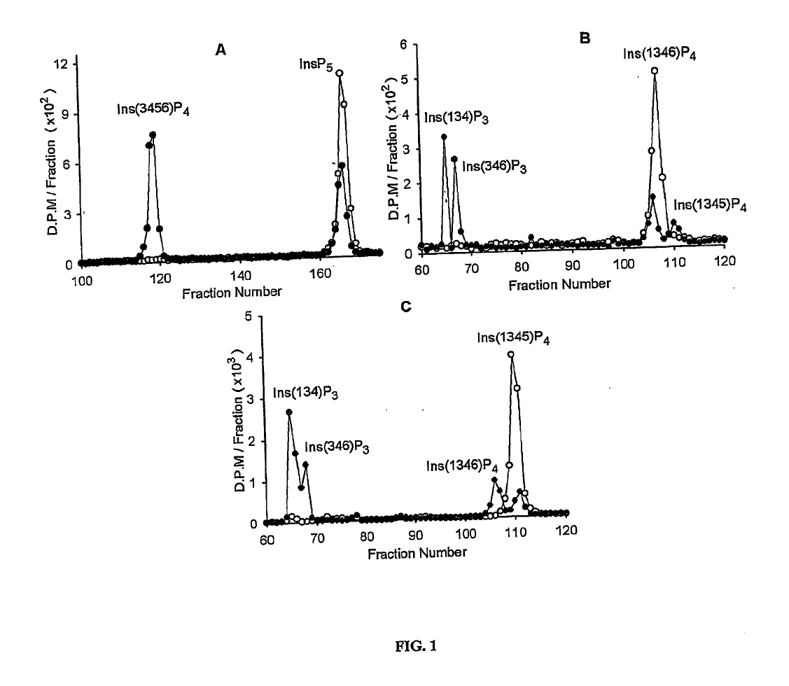

We therefore turned to the alternate possibility that Ins(3,4,5,6)P₄ synthesis might involve dephosphorylation of InsP₅; the simplest, most direct route would be by direct 1-phosphatase attack on Ins(1,3,4,5,6)P₅. While cells possess active 3-phosphatase activity against this Ins(1,3,4,5,6)P₅ (Chi et al., “Targeted deletion of Minpp1 provides new insight into the activity of multiple inositol polyphosphate phosphatase in vivo,” Mol Cell Biol 20: 6496-6507 (2000)), a 1-phosphatase activity has never been observed, even in tissues taken from 3-phosphatase “knock-out” mice [13]. Thus, we speculated that if a 1-phosphatase were to exist, we might only observe it in vitro under specialized assay conditions. We incubated the phosphokinase with 5 mM ADP, whereupon [³H]Ins(1,3,4,5,6)P₅ was dephosphorylated to an [³H]InsP₄ peak co-eluting upon HPLC with a standard of Ins(3,4,5,6)P₄ (FIG. 1A). Ins(1,4,5,6)P₄ would also elute at this point, but we eliminated this option, as the [³H]InsP₄ that was formed was a 1-kinase substrate, which Ins(1,4,5,6)P₄ is not (Caffrey et al., “Expanding Coincident Signaling by PTEN through its Inositol 1,3,4,5,6-Pentakisphosphate 3-phosphatase Activity,” FEBS Lett 499: 6-10 (2001)). Other potential products of InsP₅ dephosphorylation were all excluded by their earlier elution positions in this HPLC system (FIGS. 1B,C). In addition, we discovered (FIG. 1B) that the Ins(1,3,4)P₃ 6-kinase activity of this same enzyme (Yang et al., “Multitasking in Signal Transduction by a Promiscuous Human Ins(3,4,5,6)P₄ 1-Kinase/Ins(1,3,4)P₃ 5/6-Kinase,” Biochem J 351: 551-555 (2001)) is also reversible. That is, Ins(1,3,4,6)P₄ was dephosphorylated to Ins(1,3,4)P₃ (FIG. 1B).

The present experiments yielded several unexpected results. First, an additional, later-eluting InsP₃ accumulated following Ins(1,3,4,6)P₄ dephosphorylation, which is presumably Ins(3,4,6)P₃ (not Ins(1,4,6)P₃, see below). Second, the kinase yielded an identical pattern of InsP₃ products upon dephosphorylation of [3 H]Ins(1,3,4,5)P₄ (FIG. 1C), despite the latter not possessing a 6-phosphate. Third, Ins(1,3,4,6)P₄ and Ins(1,3,4,5)P₄ were interconverted (FIGS. 1B,C). Such “phosphomutase” activity is unprecedented in the field of inositol phosphate metabolism.

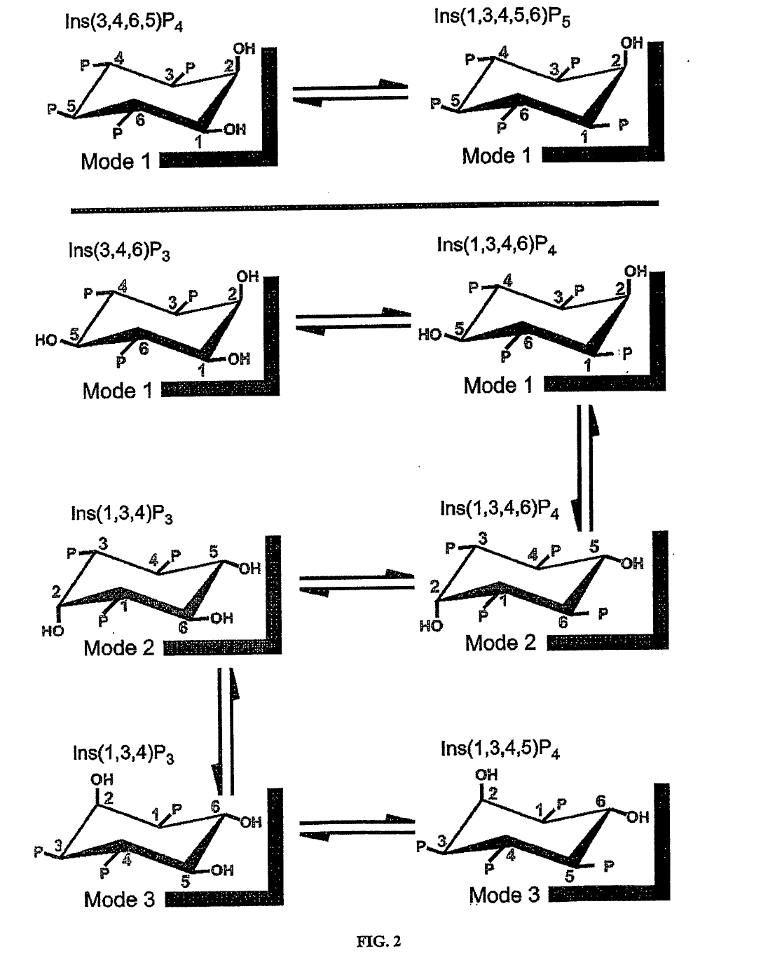

To explain kinase reversibility and its phosphomutase activity, we noted earlier studies (Wilcox et al., “Modification at C2 of myo-inositol 1,4,5-trisphosphate produces inositol trisphosphates and tetrakisphosphates with potent biological activities,” Eur J Biochem 223: 115-124 (1994)), which were more recently applied to an inositol phosphate kinase from yeast (Ongusaha et al., “Inositol hexakisphosphate in Schizosaccharomyces pombe: synthesis from Ins(1,4,5)P₃ and osmotic regulation,” Biochem J 335: 671-679 (1998)), showing that some inositol phosphates may interact with binding sites of receptors and enzymes in more than one orientation, and that an inositol phosphate may mimic another by presenting key recognition features to the site in certain binding orientations. The present data suggest a reaction pathway for the reversible kinase/phosphatase based on the idea that inositol phosphates bind in three different orientations (shown as modes 1, 2 and 3 in FIG. 2), including two modes for Ins(1,3,4)P₃. This new model (FIG. 2) predicts that Ins(3,4,6)P₃ is the later-eluting InsP₃ peak in FIGS. 1B and 1C, and further provides a novel explanation for two previously puzzling observations. The present data suggest that Ins(1,2,4)P₃ is recognized as a mode 3 substrate. Second, the ability of the kinase to phosphorylate Ins(1,3,4)P₃ at the 5- and 6-positions (Yang et al., “Multitasking in Signal Transduction by a Promiscuous Human Ins(3,4,5,6)P₄ 1-Kinase/Ins(1,3,4)P₃ 5/6-Kinase,” Biochem J 351: 551-555 (2000)).

Wilson et al., “Isolation of inositol 1,3,4-trisphosphate 5/6-kinase, cDNA cloning, and expression of recombinant enzyme,” J Biol Chem 271: 11904-11910 (1996); Abdullah et al., “Purification and characterization of inositol 1,3,4-trisphosphate 5/6-kinase from rat liver using an inositol hexakisphosphate affinity column,” J Biol Chem 267: 22340-22345 (1992)) is rationalized as reflecting two Ins(1,3,4)P₃ binding modes (FIG. 2), rather than a 5,6-cyclic intermediate (Wilson et al., “Isolation of inositol 1,3,4-trisphosphate 5/6-kinase, cDNA cloning, and expression of recombinant enzyme,” J Biol Chem 271: 11904-11910 (1996)). This model (FIG. 2), which assumes a single active site, provides the simplest explanation for the present data, but does not exclude more complex scenarios in which inositol phosphates may bind to more than one site on the protein.

Assuming that the disclosed kinase/phosphatase utilizes a single active site, there are two phosphate groups on the inositol ring that are common to all binding modes and may therefore be structural determinants for substrate recognition (coloured red in FIG. 2). The position that is reversibly phosphorylated/dephosphorylated presumably also defines substrate recognition (also coloured red in FIG. 2). This model is consistent with our other data showing the enzyme does not phosphorylate [³H]Ins(3,4)P₂, nor Ins(1,3,4,5)P₄, Ins(1,3,4,6)P₄ and Ins(1,4,5,6)P₄ (Caffrey et al., “Expanding Coincident Signaling by PTEN through its Inositol 1,3,4,5,6-Pentakisphosphate 3-phosphatase Activity,” FEBS Lett 499: 6-10 (2001); Abdullah et al., “Purification and characterization of inositol 1,3,4-trisphosphate 5/6-kinase from rat liver using an inositol hexakisphosphate affinity column,” J Biol Chem 267: 22340-22345 (1992)). While the groups colored red in FIG. 2 are likely necessary for substrate recognition, they cannot be sufficient, because Ins(1,4)P₂ itself is not phosphorylated. Therefore, other features of the inositol phosphate ligands (coloured green in FIG. 2), must contribute to recognition, and some of these additional interactions are, presumably, specific to certain binding modes.

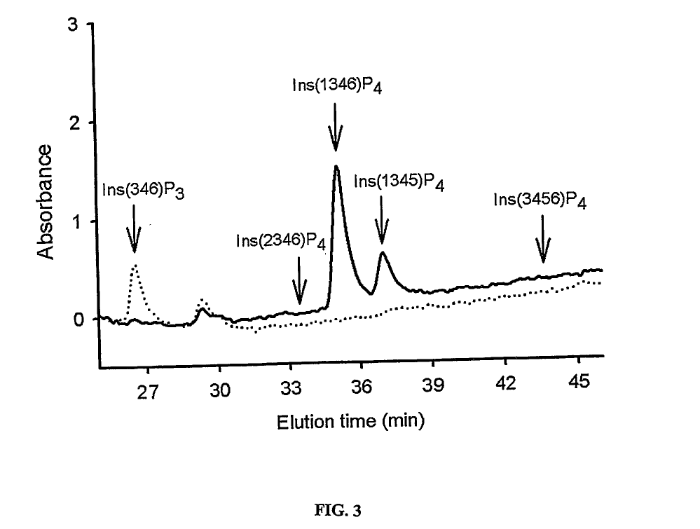

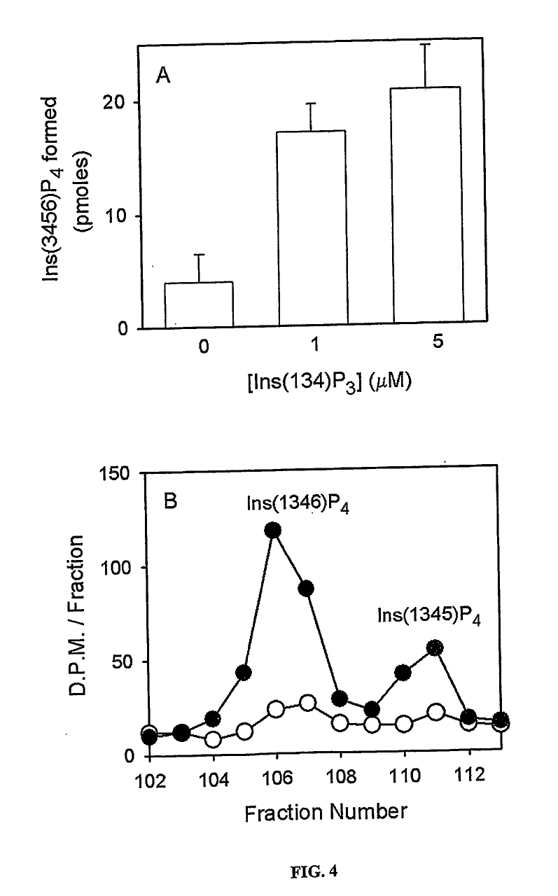

Our prediction that Ins(3,4,6)P₃ is a type 1 substrate (FIG. 2) was verified (FIG. 3) using an on-line mass detection HPLC technique (Mayr, “A novel metal-dye detection sysytem permits picomolar-range h.p.l.c. analysis of inositol polyphosphates from non-radioactively labelled cell or tissue specimens,” Biochem J 254: 585-591 (1988); Adelt et al., “Enzyme-assisted total synthesis of the optical antipodes D-myo-inositol 3,4,5-trisphosphate and D-myo-inositol 1,5,6-trisphosphate: aspects of their structure-activity relationship to biologically active inositol phosphates,” J Med Chem 42: 1262-1273 (1999)). Kinetic data indicate a Kₘ of 0.2 μM, a value close to that for Ins(1,3,4)P₃ (Yang et al., “Multitasking in Signal Transduction by a Promiscuous Human Ins(3,4,5,6)P₄ 1-Kinase/Ins(1,3,4)P₃ 5/6-Kinase,” Biochem J 351: 551-555 (2000)). We detected an InsP₄ product eluting in the position expected of Ins(1,3,4,6)P₄ (FIG. 3). Ins(1,3,4,5)P₄ was also produced (FIG. 3), although we cannot accurately quantify the Ins(1,3,4,6)P₄/Ins(1,3,4,5)P₄ ratio; the signal strength of this HPLC technique is not proportional to the number of phosphate groups (Mayr, “A novel metal-dye detection sysytem permits picomolar-range h.p.l.c. analysis of inositol polyphosphates from non-radioactively labelled cell or tissue specimens,” Biochem J 254: 585-591 (1988)) and even varies considerably between isomers containing the same number of phosphates (Adelt et al., “Enzyme-assisted total synthesis of the optical antipodes D-inyo-inositol 3,4,5-trisphosphate and D-myo-inositol 1,5,6-trisphosphate: aspects of their structure-activity relationship to biologically active inositol phosphates,” J Med Chem 42: 1262-1273 (1999)). Nevertheless, Ins(3,4,6)P₃ is clearly metabolized to Ins(1,3,4,5)P₄ (FIG. 3), consistent with the reaction pathway shown in FIG. 2. This involves dephosphorylation of Ins(1,3,4,6)P₄ to Ins(1,3,4)P₃ even though ATP (and not ADP) was added to these assays (FIGS. 2,3). Equally, the phosphorylation of Ins(1,3,4)P₃ to Ins(1,3,4,6)P₄ occurs when ADP (and not ATP) was added to the assays (FIG. 1C,3). Thus, the ability of the enzyme to act as both a kinase and a phosphatase is not entirely dictated by the ATP/ADP ratio added to the incubation media. It is possible that InSP₄ participates more directly in the phosphotransferase reactions. One way in which Ins(1,3,4)P₃ could be converted into Ins(1,3,4,6)P₄ when Ins(1,3,4,5)P₄ is the original substrate (FIG. 1C) would be for Ins(1,3,4,5)P₄ to donate its 5-phosphate group to the enzyme, forming a phosphorylenzyme intermediate, which then could transfer the phosphate to the 6-hydroxyl of Ins(1,3,4)P₃, albeit in a manner apparently dependent upon some adenine nucleotide being present. Furthermore, in incubations containing 5 μM Ins(1,3,4,5,6)P₅ and 5 mM ADP, net accumulation of Ins(3,4,5,6)P₄ was increased by up to 5-fold upon addition of 1-5 μM Ins(1,3,4)P₃ (FIG. 4A). One explanation for this result is that the newly-formed Ins(3,4,5,6)P₄ can be re-phosphorylated, but less effectively in the presence of Ins(1,3,4)P₃, a competing kinase substrate (Yang et al., “Multitasking in Signal Transduction by a Promiscuous Human Ins(3,4,5,6)P₄ 1-Kinase/Ins(1,3,4)P₃ 5/6Kinase,” Biochem J 351: 551-555 (2000)). Indeed, in these experiments there was net phosphorylation of Ins(1,3,4)P₃, but in a manner dependent upon Ins(1,3,4,5,6)P₅ (FIG. 4B), which presumably donates the phosphate group, via a phosphorylenzyme intermediate, to either Ins(1,3,4)P₃ or Ins(3,4,5,6)P₄. Whatever the explanation, our exploration of several unusual features of this enzyme have led us to uncover assay conditions that optimize Ins(3,4,5,6)P₄ synthesis (FIG. 4A). With Ins(1,3,4)P₃ present, the Vₘₐₓ for Ins(1,3,4,5,6)P₅ dephosphorylation was 82 pmol/μg protein/min, approx. 10-fold less than the Vₘₐₓ for Ins(3,4,5,6)P₄ phosphorylation by these same preparations of recombinant enzyme (Yang et al., “Multitasking in Signal Transduction by a Promiscuous Human Ins(3,4,5,6)P₄ 1-Kinase/Ins(1,3,4)P₃ 5/6-Kinase,” Biochem J 351: 551-555 (2000)), although the latter reaction was previously estimated to operate at only 5-10% of its capacity in receptor-activated cells (Tan et al., “Properties of the inositol 3,4,5,6-tetrakisphosphate 1-kinase purified from rat liver. Regulation of enzyme activity by inositol 1,3,4-trisphosphate,” J Biol Chem 272: 2285-2290 (1997)).

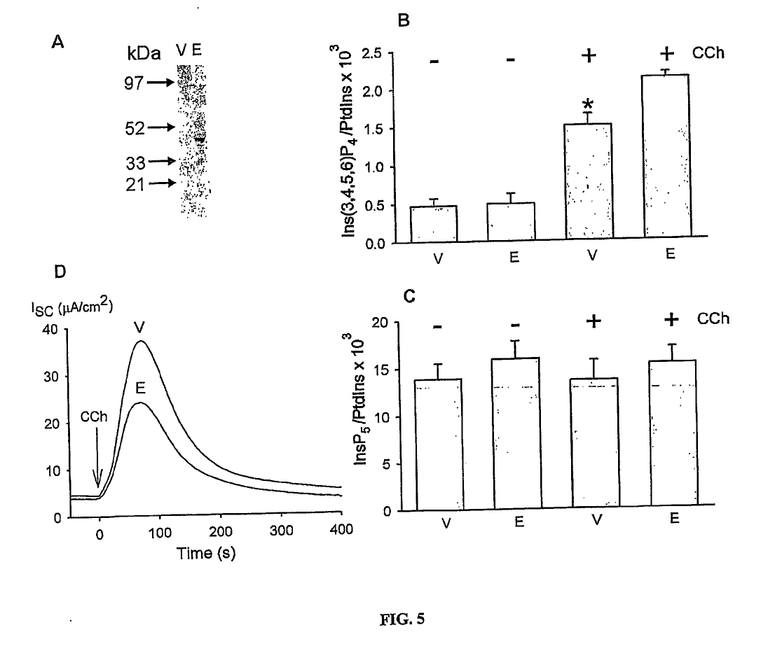

Human colonic epithelial T₈₄ cells were stably transfected with FLAG-tagged enzyme (theoretical size, 46.9 kDa). Expression was verified using anti-FLAG antibodies (FIG. 5A, 49±0.3 kDa, n=3). Despite the enzyme acting in vitro as both an Ins(3,4,5,6)P₄ 1-kinase and Ins(1,3,4,5,6)P₅ 1-phosphatase, the elevated levels of [³H]Ins(3,4,5,6)P₄ in enzyme-transfected cells indicate that phosphatase activity can predominate, specifically upon receptor activation (FIG. 5B). Levels of [³H]InsP₅ were not significantly affected by transfection (FIG. 5C).

The enhancement of the receptor-initiated Ins(3,4,5,6)P₄ response in enzyme-transfected cells was accompanied by a 40% reduction in Ca²⁺-activated Cl⁻ secretion (FIG. 5D) from an intact cell monolayer. This provides a unique validation of the signaling importance of Ins(3,4,5,6)P₄ in a physiological context. As well as regulating salt and fluid secretion, these Ca²⁺-activated Cl⁻ channels mediate cell volume homeostasis, and electrical excitability in neurons and smooth muscle (Ho et al., “Regulation of chloride channel conductance by Ins(3,4,5,6)P₄; a phosphoinositide-initiated signalling pathway that acts downstream of Ins(1,4,5)P₃,” In Frontiers in Molecular Biology: Biology of Phosphoinositides. Edited by Cockroft S. Oxford: Oxford University Press; 298-319 (2000); Frings et al., “Neuronal Ca²⁺-activated Cl⁻ channels—homing in on an elusive channel species,” Prog Neurobiol 60: 247-289 (2000)), which testifies to the wide-ranging biological impact of the present observations. Reciprocal coordination of the opposing 1-kinase/1-phosphatase reactions, catalyzed by a single enzyme, offers an alternative to general doctrine that intracellular signals are regulated by integrating multiple phosphatases and kinases (Woscholski et al., “Inositol Phosphatases: constructive destruction of phosphoinositides and inositol phosphates,” In Biology of Phosphoinositides. Edited by Cockroft S. Oxford: Oxford University Press; 320-338 (2000)). Finally, this demonstration that the InSP₅ 1-phosphatase regulates secretion (FIG. 5) is also of therapeutic interest. Up-regulation of InsP₅ 1-phosphatase in airway epithelia is expected to inhibit the gob-5 chloride channel that drives mucus secretion which, when hyper-responsive, contributes to the asthmatic condition (Nakanishi et al., “Role of gob-5 in mucus overproduction and airway hyperresponsiveness in asthma,” Proc Nat Acad Sci USA 98: 5175-5180 (2001)). Down-regulation of InsP₅ 1-phosphatase could be used to enhance Cl⁻ secretion in the therapy of cystic fibrosis (Ho et al., “Regulation of chloride channel conductance by Ins(3,4,5,6)P₄; a phosphoinositide-initiated signalling pathway that acts downstream of Ins(1,4,5)P₃,” In Frontiers in Molecular Biology: Biology of Phosphoinositides. Edited by Cockroft S. Oxford: Oxford University Press; 298-319 (2000)).

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

### REFERENCE LIST FOR EXAMPLE 1

A1. Carew M A, Yang X, Schultz C, Shears S B: Ins(3,4,5,6)P₄ inhibits an apical calcium-activated chloride conductance in polarized monolayers of a cystic fibrosis cell-line. J Biol Chem 2000, 275: 26906-26913.
A2. Nilius B. Prenen J, Voets T, Eggermont J, Bruzik K S, Shears S B et al.: Inhibition by inositoltetrakisphosphates of calcium- and volume-activated Cl⁻ currents in macrovascular endothelial cells. Pflügers Arch—Eur J Physiol 1998, 435: 637-644.
A3. Ho MWY, Carew M A, Yang X,. Shears S B: Regulation of chloride channel conductance by Ins(3,4,5,6)P₄; a phosphoinositide-initiated signalling pathway that acts downstream of Ins(1,4,5)P₃. In Frontiers in Molecular Biology: Biology of phosphoinositides. Edited by Cockroft S. Oxford: Oxford University Press; 2000:298-319.
A4. Yang X, Shears S B: Multitasking in Signal Transduction by a Promiscuous Human Ins(3,4,5,6)P₄ 1-Kinase/Ins(1,3,4)P₃ 5/6-Kinase. Biochem J 2000, 351: 551-555.
A5. Wilson M P, Majerus P W: Isolation of inositol 1,3,4-trisphosphate 5/6-kinase, cDNA cloning, and expression of recombinant enzyme. J Biol Chem 1996, 271: 11904-11910.
A6. Woscholski R, Parker P J: Inositol Phosphatases: constructive destruction of phosphoinositides and inositol phosphates. In Biology of phosphoinositides. Edited by Cockroft S. Oxford: Oxford University Press; 2000:320-338.
A7. Clapham D: How to lose your hippocampus by working on chloride channels. Neuron 2001, 29: 1-6.
A8. Ho MWY, Kaetzel M A, Armstrong D L, Shears S B: Regulation of a Human Chloride Channel: A Paradigm for Integrating Input from Calcium, CaMKII and Ins(3,4,5,6)P_{4.} J Biol Chem 2001, 276: 18673-18680.
A9. Xie W, Kaetzel M A, Bruzilc K S, Dedman J R, Shears S B, Nelson D J: Inositol 3,4,5,6-tetrakisphosphate inhibits the calmodulin-dependent protein kinase II-activated chloride conductance in T84 colonic epithelial cells. J Biol Chem 1996, 271: 14092-14097.
A10. Menniti F S, Oliver K G, Nogimori K, Obie J F, Shears S B, Putney J W, Jr.: Origins of myo-inositol tetrakisphosphates in agonist-stimulated rat pancreatoma cells. Stimulation by bombesin of myo-inositol (1,3,4,5,6) pentakisphosphate breakdown to myo-inositol (3,4,5,6) tetrakisphosphate. J Biol Chem 1990, 265: 11167-11176.
A11. Frings S, Reuter D, Kleene S J: Neuronal Ca²⁺-activated Cl⁻ channels—homing in on an elusive channel species. Prog Neurobiol 2000, 60: 247-289.
A12. Ho MWY, Shears S B, Bruzik K S, Duszyk M, French A S: Inositol 3,4,5,6-tetrakisphosphate specifically inhibits a receptor-mediated Ca²⁺-dependent Cl⁻ current in CFPAC-1 cells. Am J Physiol 1997, 272: C1160-C1168.
A13. Chi H, Yang X, Kingsley P D, O'Keefe R J, Puzas J E, Rosier R N et al.: Targeted deletion of Minpp1 provides new insight into the activity of multiple inositol polyphosphate phosphatase in vivo. Mol Cell Biol 2000, 20: 6496-6507.
A14. Caffrey J J, Darden T, Wenk M R, Shears S B: Expanding Coincident Signaling by PTEN through its Inositol 1,3,4,5,6-Pentakisphosphate 3-phosphatase Activity. FEBS Lett 2001, 499: 6-10.
A15. Wilcox R A, Safrany S T, Lampe D, Mills S J, Nahorski S R, Potter B VL: Modification at C2 of myo-inositol 1,4,5-trisphosphate produces inositol trisphosphates and tetrakisphosphates with potent biological activities. Eur J Biochem 1994, 223: 115-124.
A16. Ongusaha P P, Hughes P J, Davey J, Michell R H: Inositol hexakisphosphate in Schizosaccharomyces pombe: synthesis from Ins(1,4,5)P₃ and osmotic regulation. Biochem J 1998, 335: 671-679.
A17. Adelt S, Plettenburg O. Dallmann G, Ritter F P, Shears S B, Altenbach H-J et al.: Regiospecific Phosphohydrolases from Dictyostelium as Tools for the Chemoenzymatic Synthesis of the Enantiomers D-myo-Inositol 1,2,4-Trisphosphate and D-myo-Inositol 2,3,6-Trisphosphate: Non-Physiological, Potential Analogues of Biologically Active D-myo-Inositol 1,3,4-Trisphosphate. Bioorg Med Chem Lett 2001, 11: 2705-2708.
A18. Abdullah M, Hughes P J, Craxton A, Gigg R, Desai T, Marecek J F et al.: Purification and characterization of inositol 1,3,4-trisphosphate 5/6-kinase from rat liver using an inositol hexakisphosphate affinity column. J Biol Chem 1992, 267: 22340-22345.
A19. Mayr G W: A novel metal-dye detection sysytem permits picomolar-range h.p.l.c. analysis of inositol polyphosphates from non-radioactively labelled cell or tissue specimens. Biochem J 1988, 254: 585-591.
A20. Adelt S, Plettenburg O, Stricker R, Reiser G, Altenbach H-J, Vogel G: Enzyme-assisted total synthesis of the optical antipodes D-myo-inositol 3,4,5-trisphosphate and D-myo-inositol 1,5,6-trisphosphate: aspects of their structure-activity relationship to biologically active inositol phosphates. J Med Chem 1999, 42: 1262-1273.
A21. Tan Z, Bruzik K S, Shears S B: Properties of the inositol 3,4,5,6-tetrakisphosphate 1-kinase purified from rat liver. Regulation of enzyme activity by inositol 1,3,4-trisphosphate. J Biol Chem 1997, 272: 2285-2290.
A22. Nakanishi A, Morita S, Iwashita H, Sagiya Y, Ashida Y, Shirafuji H et al.: Role of gob-5 in mucus overproduction and airway hyperresponsiveness in asthma. Proc Nat Acad Sci USA 2001, 98: 5175-5180.
A23. Mills S J, Potter B VL: Synthesis of D- and L-myo-inositol 1,4,6-trisphosphate, regioisomers of a ubiquitous second messenger. J Org Chem 1996, 61: 8980-8987.
A24. Zhang T, Caffrey J J, Shears S B: The Transcriptional Regulator, Arg82, is a Hybrid Kinase with both Monophosphoinositol- and Diphosphoinositol-Polyphosphate Synthase Activity. FEBS Lett 2001, 494: 208-212.
A25. Erneux C, De Smedt F, Moreau C, Rider M, Communi D: Production of recombinant human brain type I inositol-1,4,5-trisphosphate 5-phosphatase in Escherichia coli. Lack of phosphorylation by protein kinase C. Eur J Biochem 1995, 234: 598-602.

### Example 2

ABSTRACT

ClC Cl⁻ channels in endosomes, synaptosomes, lysosomes and beta-cell insulin granules provide charge-neutralization support for the functionally-indispensable acidification of the luminal interior by electrogenic H⁺-ATPases (Jentsch et al., 2002 Physiol. Rev. 82 503). Regulation of ClC activity is, therefore, of widespread biological significance (Forgac, 1999 J. Biol. Chem. 274 12951). We now ascribe just such a regulatory function to the increases in cellular levels of Ins(3,4,5,6)P₄ that inevitably accompany activation of the ubiquitous Ins(1,4,5)P₃ signaling pathway. We used confocal imaging to record insulin granule acidification in single mouse pancreatic β-cells. Granule acidification was reduced by perfusion of single cells with 10 μM Ins(3,4,5,6)P₄ (the concentration following receptor-activation), whereas at 1 μM (“resting” levels), Ins(3,4,5,6)P₄ was ineffective. This response to Ins(3,4,5,6)P₄ was not mimicked by 100 μM Ins(1,4,5,6)P₄ nor by 100 μM Ins(1,3,4,5,6)P₅. Ins(3,4,5,6)P₄ did not affect granular H⁺-ATPase activity, nor H⁺ leak, indicating Ins(3,4,5,6)P₄ instead inhibited charge neutralization by ClC. The Ins(3,4,5,6)P₄-mediated inhibition of vesicle acidification reduced exocytic release of insulin, as determined by whole-cell capacitance recordings. This may impinge upon type 2 diabetes etiology. Regulatory control over vesicle acidification by this negative signaling pathway in other cell types should be considered.

Introduction

Elevations in Ins(3,4,5,6)P₄ levels are ubiquitously coupled to stimulus-dependent activation of the Ins(1,4,5)P₃ signaling pathway (B1;B2). When this observation was first made, Ins(3,4,5,6)P₄ had no known physiological function, and was deemed to be an “orphan” signal (B2). Subsequently, Ins(3,4,5,6)P₄ was shown to inhibit the conductance through Ca²⁺-activated Cl⁻ channels (CLCA) in the plasma membrane (B3-B5). However, this signaling paradigm has, to date, seemed restricted to epithelial salt and fluid secretion (B6;B7). This has raised the question as to whether, in other cell types, there can be wider biological significance to receptor-dependent changes in Ins(3,4,5,6)P₄ levels.

There are Cl⁻ channels that are different from CLCA which are expressed in intracellular vesicles, such as endosomes, (B8), synaptosomes (B8) and insulin granules in β-cells (B9), where they co-localize with H⁺-ATPases (B10-B12). The acidification of the vesicular interior by these H⁺-ATPases serves a number of important functions, including modulation of certain ligand-protein interactions during endocytosis, enzyme targeting, coupled transport of small molecules, and optimization of proteolytic activities of, for example, prohormone processing enzymes (B13). It has also been proposed that luminal acidification drives the priming of insulin granules so that they become competent to fuse with the plasma membrane and release their cargo (B9). This particular paradigm could be more widely applicable to endocrine and neurotransmitter release.

If the vesicle acidification driven by the electrogenic H⁺-ATPase were not to be charge compensated, a large transmembrane electrical gradient would develop, and this would then prevent further H⁺ pumping, even before a substantial acidification of the vesicular interior could take place (B8;B12;B13). Cl⁻ flux into the vesicles provides the electrical neutralization that is necessary to prevent an inhibitory electrochemical gradient from forming (B8;B12;B13). The molecular nature of the channels that are responsible for this Cl⁻ flux has been ascertained; they are members of the ClC family (B8;B9;B11). Unlike CLCA, the intracellular ClC channels are not generally considered to be Ca²⁺-activated (B8). Indeed, the ClC channels show no sequence homology with any of the Ins(3,4,5,6)P₄-sensitive CLCA channels that have been cloned (B5;B 14). Nevertheless, we have now investigated if there is a functionally-significant interaction of Ins(3,4,5,6)P₄ with ClC channels; our model system has been mouse beta-cell insulin granules, which were recently ascertained to contain ClC3 (B9). The results that we have obtained expand the biological significance of Ins(3,4,5,6)P₄.

Experimental Procedures

The inositol phosphates used in this study were all purchased from CellSignals Inc (Lexington, Ky.). Insulin granule pH were monitored by combining the patch-clamp technique with confocal imaging. Pancreatic β-cells, isolated and cultured on glass coverslips for 2-24 h, were voltage clamped at −70 mV, as previously described (B9). LysoSensor-Green™ DND-189 (1 μM; Molecular Probes, Leiden, The Netherlands) was added during the last 30 min of cell culture and was also included in the extracellular buffer (B9) that continuously perfused the experimental chamber. All LSG experiments were performed at 20° C. to prevent exocytosis that otherwise interfered with the optical measurements (15). LSG fluorescence was excited using the 488 nm-line of a Zeiss 510 confocal microscope. The emitted light was collected with a 63×/1.3 NA oil objective and a >505 nm filter. Laser scanning was performed with low pixel resolution (128×128) and with 6 s intervals to minimize photo-bleaching. After gigaseal formation, it was ascertained that LSG fluorescence was stable for at least 30 s before the experiment commenced. The intracellular electrode solution contained: 125 mM K-glutamate, 10 mM KCl, 1 0 mM NaCl, 1 mM MgCl₂, 5 mM HEPES, 3 mM Mg-ATP, 0.1 mM cAMP, 10 mM EGTA and 7 mM CaCl₂ (˜0.4 μM free [Ca²⁺]_{I}; pH 7.2 with KOH). In some experiments, marked “no calcium”, there was no added Ca²⁺, and the concentration of EGTA was 50 μM. Whole-cell capacitance measurements (at 33° C.) were made with the cells clamped at −70 mV, and the standard whole-cell configuration was established at t=0. Capacitance recordings were made using an EPC-7 patch-clamp amplifier equipped with the software Pulse (version 8.4 and later) and the X-chart extension (HEKA Elektronik, Lambrecht/Pfalz, Germany) (B9).

Results and Discussion

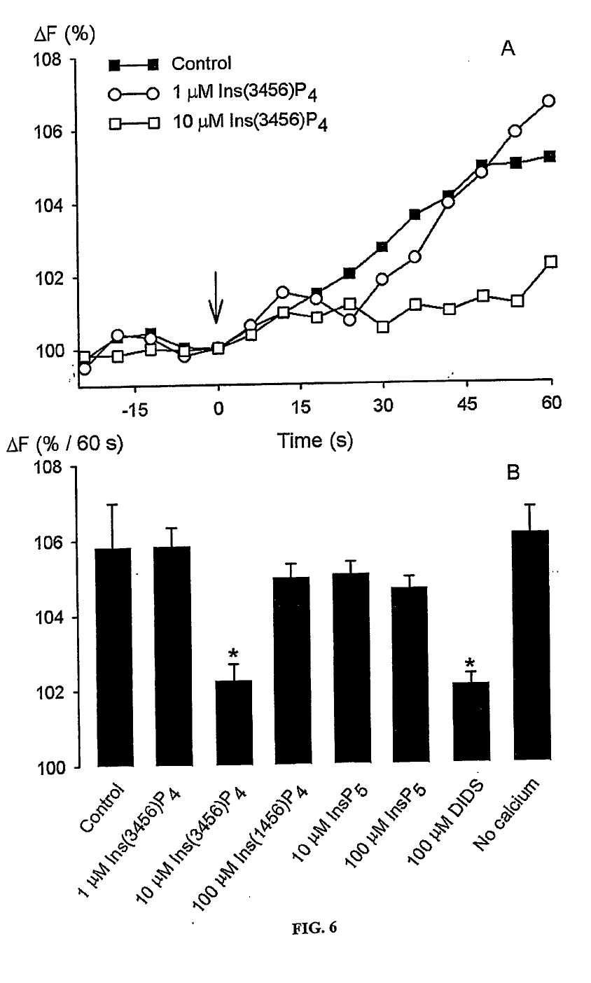

Since insulin granules comprise the major acidic compartment in murine α-cells, LSG fluorescence, measured by confocal imaging, was employed to monitor changes in intragranular pH (B9). Immediately after “intracellular” medium was perfused into a single cell through a patch-clamp pipette (“control” trace in FIG. 6A), a progressive increase in fluorescence intensity was observed, reflecting an increased acidification of the granule interior. As indicated in the Introduction to Example 2, granule acidification requires charge compensation through the action of an active Cl⁻ conductance. This is verified by the demonstration that acidification was inhibited either by a generic Cl⁻ channel blocker, DIDS (FIG. 6B), or by lowering the cytoplasmic Cl⁻ concentration (B9), or by perfusing the cells with functionally-inactivating anti-ClC3 antibodies (B9). Next, Ins(3,4,5,6)P₄ was perfused into the cell at a concentration (10 μM) typically attained after several minutes receptor activation of phospholipase C (B6). The Ins(3,4,5,6)P₄ reduced by approximately 65% the rate of acidification of the granular interior (FIGS. 6A,B). This is a specific effect; neither 100 μM Ins(1,4,5,6)P₄ nor 100 μM Ins(1,3,4,5,6)P₅ affected vesicle acidification (FIG. 6B). A concentration of Ins(3,4,5,6)P₄ equivalent to that in resting cells (1 μM), was ineffective (FIGS. 6A,B), indicating that, in vivo, this new role for Ins(3,4,5,6)P₄ is dependent upon stimulus-mediated increases in its concentration.

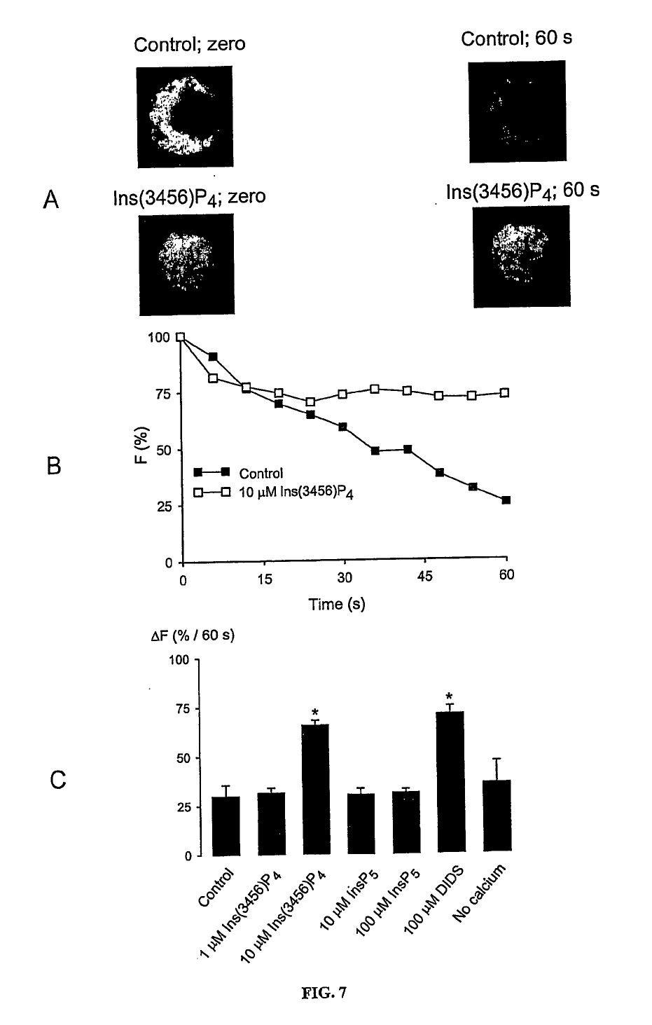

Another approach to studying the relevance of ClC channels to transmembrane H⁺ movements is to discharge the H⁺ gradient across the membrane of the insulin granule with the protonophore CCCP (FIG. 7). Protonophonic H+ flux is electrogenic, such that collapse of the transmembrane pH gradient requires operation of the Cl⁻ channel charge shunt (B9). Thus, loss of H⁺ from the granules is inhibited upon blockade of the Cl⁻ conductance by DIDS (FIG. 7B and ref (B9)). A quantitatively similar, inhibitory effect upon CCCP-induced H⁺ flux was observed when the cells were perfused with 10 μM Ins(3,4,5,6)P₄ (FIG. 7). Note that, in these experiments, the intracellular medium contained ATP to support continued operation of the H⁺-ATPase. If Ins(3,4,5,6)P₄ had inhibited the H⁺-ATPase, the polyphosphate would have been expected to accelerate the rate of dissipation of the pH gradient; this is the opposite of the result that was actually obtained. Equally, the failure of the H⁺ gradient to discharge in the presence of 10 μM Ins(3,4,5,6)P₄ (FIG. 7) indicates that the polyphosphate did not increase granule H⁺ leak. We therefore conclude that Ins(3,4,5,6)P₄ inhibits vesicle acidification (FIG. 6) by regulating the granule Cl⁻ conductance. In control experiments, we added 100 μM Ins(1,3,4,5,6)P₅, and this had no effect on the rate at which CCCP promoted H⁺ efflux out of the granule (FIG. 7). Resting levels of Ins(3,4,5,6)P₄ (1 μM) also failed to affect CCCP-induced collapse of the pH gradient.

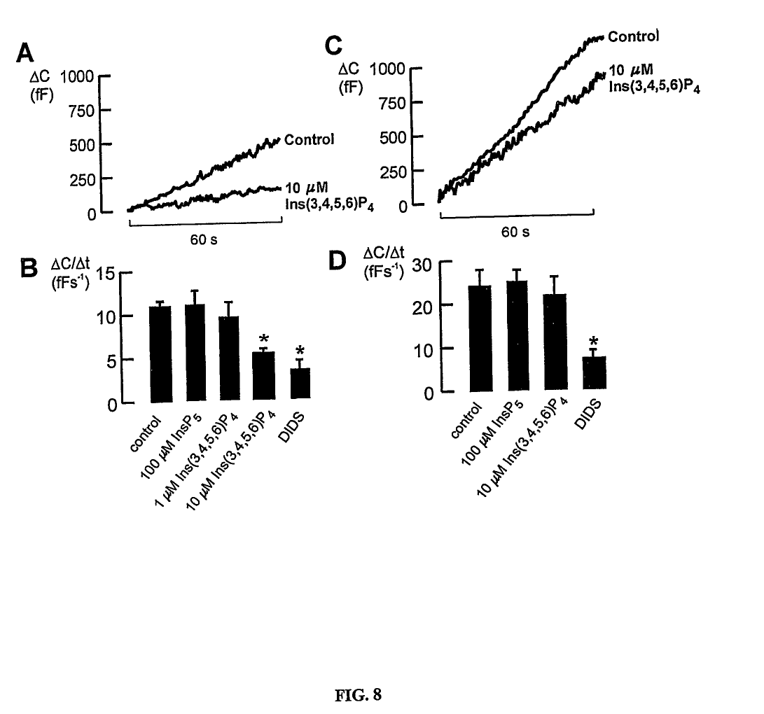

The degree of insulin granule acidification in pancreatic β-cells has been proposed to affect their fusogenic potential, thereby influencing the rate of insulin secretion by exocytosis (B9;B16). So we next investigated if the interaction of Ins(3,4,5,6)P₄ with insulin granule Cl⁻ channels affected exocytosis, which we recorded by monitoring capacitance of β-cells with the whole-cell patch-clamp technique (B16). When the cell was perfused with an “intracellular” medium containing 0.4 μM [Ca²⁺]_{free}, (i.e the cytosolic [Ca²⁺] attained after glucose levels are elevated (B17)) a progressive increase in cell capacitance was observed, reflecting Ca²⁺-dependent activation of granule exocytosis (FIG. 8A). Ins(3,4,5,6)P₄ (10 μM) inhibited exocytosis by approximately 50% (FIGS. 8A,B). Since 1 μM Ins(3,4,5,6)P₄ was ineffective (FIGS. 8A,B), inhibition of exocytosis by Ins(3,4,5,6)P₄ in vivo can be anticipated to be stimulus-dependent. Ins(1,3,4,5,6)P₅ (100 μM) had no effect upon exocytosis (FIGS. 8A,B). When cells were perfused with intracellular medium containing 1.5 μM [Ca²⁺]_{free}, the rate of exocytosis was about 2-fold faster than was obtained with 0.4 μM [Ca²⁺]_{free} (FIG. 8). Interestingly, in this condition Ins(3,4,5,6)P₄ did not affect the rate of exocytosis (FIGS. 8C,D). Thus, large intracellular Ca²⁺ transients offer an opportunity for dynamic by-pass of the inhibition of exocytosis by Ins(3,4,5,6)P₄. Exocytosis that is stimulated by smaller Ca²⁺ transients (<0.4 μM), such as those elicited by glucose (B17), would be more sensitive to inhibition by Ins(3,4,5,6)P₄, according to our data (FIGS. 8A,B).

Prior to this report, Ins(3,4,5,6)P₄ was only known to be physiologically active as an inhibitor of salt and fluid secretion from epithelial cells (B6;B7) through modulation of at least some members of the CLCA family (B3;B5;B14). Yet, stimulus-dependent, Ins(1,4,5)P₃-coupled increases in Ins(3,4,5,6)P₄ levels are a ubiquitous phenomenon (B2). Now we have increased our understanding of the biological significance of Ins(3,4,5,6)P₄, by showing that is also regulates insulin granule acidification. We conclude that charge neutralization by ClC3 (the species of ClC in insulin granules (B9)) is the target of Ins(3,4,5,6)P₄ action. It is reasonable to expect that future experiments will still further expand the signaling repertoire of Ins(3,4,5,6)P₄; ClC3 is highly conserved and widely expressed, and is important to endocytosis, especially in neurons and neuroendocrine cells, and ClC3 is also necessary for development of the retina and hippocampus (B8;B11). Equally, the ClC channel on insulin granules must now be considered a regulated signaling entity, rather than being subservient to vesicle acidification. Our work also confirms and extends the hypothesis (B9;B16) that the degree of insulin granule acidity is a decisive factor in the regulation of exocytosis.

The new action of Ins(3,4,5,6)P₄ that we describe in the current study differs in one important aspect from its ability to inhibit epithelial salt and fluid secretion, which Ins(3,4,5,6)P₄ achieves by preventing Cl⁻ conductance through CLCA from being activated by either calmodulin-dependent protein kinase II (B3;B18) or by Ca²⁺ itself (B5). The ClC3 Cl⁻ channels present on insulin granules (B9) are not generally considered to be activated by Ca²⁺ (B8). Indeed, lowering the [Ca²⁺]_{free} from 0.4 μM to below 0.1 μM had no impact upon granule acidification (FIGS. 6 and 7). Treatment of the cells with W-7, a calmodulin antagonist, was similarly without effect. Thus, Ins(3,4,5,6)P₄ inhibits granule acidification by a mechanism that does not depend upon Ca²⁺-signaling.

New perspectives to beta-cell research arise from our discovery that inhibition of granule acidification by Ins(3,4,5,6)P₄ is associated with down-regulation of insulin secretion. For example, it has not previously been considered that an inositol phosphate signaling process downstream of Ins(1,4,5)P₃ might have an inhibitory influence upon insulin granule exocytosis. Ins(3,4,5,6)P₄ is likely a homeostatic brake on Ins(1,4,5)P₃-activated insulin secretion initiated both by glucose (B21) and by parasympathetic innervation of the pancreas (B22). In particular, accumulation of the slowly-metabolized (B6) Ins(3,4,5,6)P₄ signal following sustained glucose-dependent activation of the Ins(1,4,5)P₃ signaling pathway (B21) could contribute to hyperglycemia-dependent refractoriness of β-cells (B23), which typifies type 2 diabetes etiology (B24). It should also be noted that, independently of the status of the Ins(1,4,5)P₃ signaling pathway, the cellular level of Ins(3,4,5,6)P₄ will be elevated in response to a decrease in the prevailing ATP/ADP ratio (1325). This phenomenon adds an extra facet to our understanding of the many mechanisms by which the ATP/ADP status of pancreatic β-cells may influence insulin secretion (B26).

### REFERENCE LIST FOR EXAMPLE 2

B1. Ho, M. W., Yang, X., Carew, M. A., Zhang, T., Hua, L., Kwon, Y.-U., Chung, S.-K., Adelt, S., Vogel, G., Riley, A. M., Potter, B. V. L., and Shears, S. B. (2002) Current Biology 12,477-482
B2. Menniti, F. S., Oliver, K. G., Putney, J. W., Jr., and Shears, S. B. (1993) Trends.Biochem.Sci. 18, 53-56
B3. Ho, M. W. Y., Kaetzel, M. A., Armstrong, D. L., and Shears, S. B. (2001) J. Biol. Chem. 276, 18673-18680
B4. Xie, W., Solomons, K. R. H., Freeman, S., Kaetzel, M. A., Bruzik, K. S., Nelson, D. J., and Shears, S. B. (1998) J.Physiol. (Lond.) 510, 661-673
B5. Ismailov, 1. I., Fuller, C. M., Berdiev, B. K., Shlyonsky, V. G., Benos, D. J., and Barrett, K. E. (1996) Proc. Nat. Acad. Sci. USA 93, 10505-10509
B6. Vajanaphanich, M., Schultz, C., Rudolf, M. T., Wasserman, M., Enyedi, P., Craxton, A., Shears, S. B., Tsien, R. Y., Barrett, K. E., and Traynor-Kaplan, A. E. (1994) Nature 371, 711-714
B7. Carew, M. A., Yang, X., Schultz, C., and Shears, S. B. (2000) J. Biol. Chem. 275, 26906-26913
B8. Jentsch, T. J., Stein, V., Weinreich, F., and Zdebik, A. A. (2002) Physiol. Rev. 82, 503-568
B9. Barg, S., Huang, P., Eliasson, L., Nelson, D. J., Obermüller, S., Rorsman, P., Thévenod, F., and Renström, E. (2001) J. Cell. Sci. 114, 2145-2154
B10. Jentsch, T. J., Friedrich, T., Schriever, A., and Yamada, H. (1999) Pflügers Arch.—Eur. J. Physiol. 437, 783-795
B11. Stobrawa, S. M., Breiderhoff, T., Takamori, S., Engel, D., Schweizer, M., Zdebik, A. A., Bösl, M. R., Ruether, K., Jahn, H., Draguhn, A., Jahn, R., and Jentsch, T. J. (2001) Neuron 29, 185-196
B12. Al-Awqati, Q., Barasch, J., and Landry, D. (1992) J. Exp. Biol. 172, 245-266
B13. Nishi, T. and Forgac, M. (2002) Nat Rev Mol Cell Biol 3, 94-103
B14. Zhang, H., McCarthy, M., Barrett, K. E., Yankaskas, J. R., Benos, D. J., and Fuller, C. M. (2001) Ped. Pulm. Suppl. 21, A88
B15. Renström, E., Eliasson, L., Bokvist, K., and Rorsman, P. (1996) J. Physiol. (Lond.) 494, 41-52
B16. Barg, S., Renström, E., Berggren, P.-O., Bertorello, A., Bokvist, K., Braun, M., Eliasson, L., Holmes, W. E., Köhler, M., Rorsman, P., and Thévenod, F. (2000) Proc. Nat. Acad. Sci. USA 96, 5539-5544
B17. Jonas, J.-C., Gilon, P., and Henquin, J.-C. (1998) Diabetes 47, 1266-1273
B18. Xie, W., Kaetzel, M. A., Bruzik, K. S., Dedman, J. R., Shears, S. B., and Nelson, D. J. (1996) J. Biol. Chem. 271, 14092-14097
B19. Zawalich, W. S., Zawalich, K. C., and Kelley, G. G. (1996) Pflugers Arch. Eur. J. Physiol. 432, 589-596
B20. Ishihara, H., Wada, T., Kizuki, N., Asano, T., Yazaki, Y., Kikuchi, M., and Oka, Y. (1999) Biochem. Biophys. Res. Commun. 254, 77-82
B21. Biden, T. J., Peter-Riesch, B., Schlegel, W., and Wollheim, C. B. (1987) J. Biol. Chem. 262, 3567-3571
B22. Gilon, P. and Henquin, J.-C. (2001) Endocrine Reviews 22, 565-604
B23. Meyer, J., Sturis, J., Katschinski, M., Arnold, R., Goke, B., and Byrne, M. M. (2002) Am. J. Physiol. Endocrinol. Metab. 282, E917-E922
B24. Kilpatrick, E. D. and Robertson, R. P. (1998) Diabetes 47, 606-611
B25. Oliver, K. G., Putney, J. W., Jr., Obie, J. F., and Shears, S. B. (1992) J. Biol. Chem. 267, 21528-21534
B26. Lang, J. (1999) Eur. J. Biochem. 259, 3-17

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method of reducing salt, fluid or mucous secretion in a subject, comprising increasing the activity of inositol 1,3,4,5,6 pentakisphosphate 1-phosphatase in the subject.

2. A method of treating a disease that is exacerbated by salt, fluid or mucous secretion, comprising increasing the activity of inositol 1,3,4,5,6 pentakisphosphate 1-phosphatase in a subject having a disease that is exacerbated by mucous, whereby salt, fluid or mucous secretion is reduced and the disease is treated.

3. The method of claim 1 wherein the step of increasing the activity of inositol 1,3,4,5,6 pentakisphosphate 1-phosphatase comprises over-expressing inositol 1,3,4,5,6 pentakisphosphate 1-phosphatase.

4. The method of claim 1 wherein the step of increasing the activity of inositol 1,3,4,5,6 pentakisphosphate 1-phosphatase comprises adiministering to the subject a compound that activates inositol 1,3,4,5,6 pentakisphosphate 1-phosphatase.

5. The method of claim 2, wherein the disease is selected from the group consisting of asthma, bronchitis and the common cold.

6. A method of increasing salt and fluid secretion in a subject, comprising decreasing the activity of inositol 1,3,4,5,6 pentakisphosphate 1-phosphatase in the subject.

7. A method of treating a disease that is treated by increased salt and fluid secretion, comprising decreasing the activity of inositol 1,3,4,5,6 pentakisphosphate 1-phosphatase in a subject having a disease that is treated by increased salt and fluid secretion, whereby salt and fluid secretion is increased and the disease is treated.

8. The method of claim 6 wherein the step of decreasing the activity of inositol 1,3,4,5,6 pentakisphosphate 1-phosphatase comprises reducing the expression of inositol 1,3,4,5,6 pentakisphosphate 1-phosphatase.

9. The method of claim 6 wherein the step of decreasing the activity of inositol 1,3,4,5,6 pentakisphosphate 1-phosphatase comprises administering to the subject a compound that decreases the activity of inositol 1,3,4,5,6 pentakisphosphate 1-phosphatase.

10. The method of claim 7, wherein the disease is cystic fibrosis.

11. A method of decreasing chloride secretion from calcium-activated chloride channels in a cell, comprising decreasing inositol 3,4,5,6 phosphate kinase activity in the cell.

12. A method of decreasing chloride secretion from calcium-activated chloride channels in a cell, comprising increasing inositol 1,3,4,5,6 pentakisphosphate 1-phosphatase activity in the cell.

13. The method of claim 11, wherein the cell is selected from the group consisting of an epithelial cell, a neuron and a smooth muscle cell.

14. A method of increasing chloride secretion from calcium-activated chloride channels in a cell, comprising increasing inositol 3,4,5,6 phosphate kinase activity in the cell.

15. A method of increasing the activity of calcium-activated chloride channels in a cell, comprising decreasing inositol 1,3,4,5,6 pentakisphosphate 1-phosphatase activity in the cell.

16. The method of claim 14, wherein the cell is selected from the group consisting of an epithelial cell, a neuron and a smooth muscle cell.

17. A method of reducing inositol 3,4,5,6 tetrakisphosphate 1-phosphate-mediated inhibition of insulin release, comprising decreasing the activity of inositol 1,3,4,5,6 pentakisphosphate 1-phosphatase in the subject.

18. A method of treating a disease that is exacerbated by inhibition of insulin secretion, comprising decreasing the activity of inositol 1,3,4,5,6 pentakisphosphate 1-phosphatase in a subject having a disease that is exacerbated by inhibition of insulin secretion, whereby the inhibition of insulin secretion is reduced and the disease is treated.

19. The method of claim 18 wherein the disease is type 2 diabetes.

20. The method of claim 18 wherein the step of decreasing the activity of inositol 1,3,4,5,6 pentakisphosphate 1-phosphatase comprises reducing expression of inositol 1,3,4,5,6 pentakisphosphate 1-phosphatase.

21. The method of claim 18 wherein the step of decreasing the activity of inositol 1,3,4,5,6 pentakisphosphate 1-phosphatase comprises administering to the subject a compound that inhibits inositol 1,3,4,5,6 pentakisphosphate 1-phosphatase.

22. A method of increasing inositol 3,4,5,6 tetrakisphosphate 1-phosphate-mediated inhibition of insulin release, comprising increasing the activity of inositol 1,3,4,5,6 pentakisphosphate 1-phosphatase in the subject.

23. A method of treating a disease that is treated by increasing levels of inositol 3,4,5,6 tetrakisphosphate thereby modulating inositol 3,4,5,6 tetrakisphosphate 1-phosphate-regulated chloride channels in intracellular vesicles, comprising decreasing the activity of inositol 1,3,4,5,6 pentakisphosphate 1-phosphatase in a subject having a disease that is treated by decreased inositol 1,3,4,5,6 pentakisphosphate 1-phosphatase activity, whereby the disease is treated.

24. A method of treating a disease that is treated by decreasing levels of inositol 3,4,5,6 tetrakisphosphate thereby modulating inositol 3,4,5,6 tetrakisphosphate 1-phosphate-regulated chloride channels in intracellular vesicles, comprising increasing the activity of inositol 1,3,4,5,6 pentakisphosphate 1-phosphatase in a subject having a disease that is treated by increased inositol 1,3,4,5,6 pentakisphosphate 1-phosphatase activity, whereby the disease is treated.

25. The method of claim 23 wherein the step of modulating the activity of inositol 3,4,5,6 tetrakisphosphate 1-phosphate-regulated channels comprises altering the expression of inositol 1,3,4,5,6 pentakisphosphate 1-phosphatase.

26. The method of claim 23 wherein the step of modulating the activity of inositol 1,3,4,5,6 pentakisphosphate 1-phosphatase comprises administering to the subject a compound that decreases the activity of inositol 1,3,4,5,6 pentakisphosphate 1-phosphatase.

27. A method of decreasing chloride secretion from calcium-activated chloride channels in an intracellular vesicle, comprising decreasing inositol 3,4,5,6 phosphate kinase activity in the cell.

28. A method of increasing chloride secretion from calcium-activated chloride channels in an intracellular vesicle, comprising increasing inositol 3,4,5,6 phosphate kinase activity in the intracellular vesicle.

29. A method of increasing the activity of calcium-activated chloride channels in an intracellular vesicle, comprising decreasing inositol 1,3,4,5,6 pentakisphosphate 1-phosphatase activity in the intracellular vesicle.


SEQUENCE LISTING (2 sequences)

SEQ ID NO: 1 - DNA, 3020 bp, Homo sapiens
gcggccgccagactcggcctgtgggcgatttcctccggacccaggctccccgcccgaggaggaagatgcagacctttctgaaagggaagagagttggctactggctgagcgagaagaaaatcaagaagctgaatttccaggccttcgccgagctgtgcaggaagcgagggatggaggttgtgcagctgaaccttagccggccgatcgaggagcagggccccctggacgtcatcatccacaagctgactgacgtcatccttgaagccgaccagaatgatagccagtccctggagctggtgcacaggttccaggagtacatcgatgcccaccctgagaccatcgtcctggacccgctccctgccatcagaaccctgcttgaccgctccaagtcctatgagctcatccggaagattgaggcctacatggaagacgacaggatctgctcgccacccttcatggagctcacgagcctgtgcggggatgacaccatgcggctgctggagaagaacggcttgactttcccattcatttgcaaaaccagagtggctcatggcaccaactctcacgagatggctatcgtgttcaaccaggagggcctgaacgccatccagccaccctgcgtggtccagaatttcatcaaccacaacgccgtcctgtacaaggtgttcgtggttggcgagtcctacaccgtggtccagaggccctcactcaagaacttctccgcaggcacatcagaccgtgagtccatcttcttcaacagccacaacgtgtcaaagccggagtcgtcatcggtcctgacggagctggacaagatcgagggcgtgttcgagcggccgagcgacgaggtcatccgggagctctcccgggccctgcggcaggcactgggcgtgtcactctttggcatcgacatcatcatcaacaaccagacagggcagcacgccgtcattgacatcaatgccttcccaggctacgagggcgtgagcgagttcttcacagacctcctgaaccacatcgccactgtcctgcagggccagagcacagccatggcagccacaggggacgtggccctgctgaggcacagcaagcttctggccgagccggcgggcggcctggtgggcgagcggacatgcagcgccagccccggctgctgcggcagcatgatgggccaggacgcgccctggaaggctgaggccgacgcgggcggcaccgccaagctgccgcaccagagactcggctgcaacgccggcgtgtcgcccagcttccagcagcattgtgtggcctccctggccaccaaggcctcctcccagtagccacggagccgggacccagaggggcagcgcagggcgcagagcacacccgctgggccagcagctcccaacggcgatgctactactaagaatccccagtgatctgattcttctgttttttaatttttaacctgattctctgatgtcatgatctaaatgaggggtagtgggggatggaagagagtaccaggtggtccaccgttggggagtggggccgtccgcctgctctctactgtgcagacctcctaactgagtttacacacgcttgtgtttgcaacactaggtctggatgggaggtgaggggtgtgcgtatggctgccatgccagtgtctgtgcacatccctgtctgttggtctccatggccactgtggaccgggacccttgggggaagcctgcccatgtcgggctgtgggaggctgatcggtgcatgtgagagtggcttcccttctgcctgactccccactccctgacctgccccttccttgtttttcctcctactggtctccaccaaggctttgttagcccccaccctgcctggtgtgcagctaacccctccctccccacagccagaggaggccacagacccctcaggggagttccgcgctggggtctgggctgtgctccctcactaaagggaaggaaaggaagctgggcgtcctctgggccccccaacacacgtcccatttagccctgcacagcggtctccttcccctaagccagcactgctgctccctggagcccgggaaggaggctgcctggctggaggcccgagccgatggcgcctgtgctgaggatttgtgctgtgatttgggcaaatcattccaggtctttgggcctccacccccttgtctctagtggacatttgagatcagagagcaccacagggctggctttgtgccctaacccctgggatgcagcctgcctttccataaagtcacctaggtgaggataggcgcgggagcctcggcatgacaccatggagatcggggccctcttcccagtgggttcactccttttcacacctgctgggtccctcctcacccagcaggcctggtccacctctcattgcaagccccaagcactgagccagcaggtgccagggagccacccgccccccatcgcttctgcacacctcagactcaccccatcaccttggcagcaaagcactggctctgccgtctgacccctgatccaggcagccccctctgcagagaaaagggttggggagaagcctctgcagtcctggaagatgtggggtgctgggtgagaggcatcagcccccacaagtatgtttttgtgtcttaagatagcagtttactttgaaaaagtgaaaaaggcttccgggctgtcctctgcccagtgagatggaggacgctagagaaagtgctgagtgtcccgagagaggcccccgagccagtgcatggaggtccttcggcctggctcagctgggctgcaggatgcccactttgaggagggaggcacagggcttgggcgaggggcagaggccatcagaactgcccggcttttttggaaactgaggacccaacaactaaccacgtttacacgacttgagttttgaaccccgattaatgtctgtacgtcacctttcctagttctgaccctgagccctggggaacaggaaagcgtggctggcctcttgcactgctttgtctccaaaataaactactgaaatcaaaacctcgtgccgaattc

SEQ ID NO: 2 - protein, 414 aa, Homo sapiens
MQTFLKGKRVGYWLSEKKIKKLNFQAFAELCRKRGMEVVQLNLSRPIEEQGPLDVIIHKLTDVILEADQNDSQSLELVHRFQEYIDAHPETIVLDPLPAIRTLLDRSKSYELIRKIEAYMEDDRICSPPFMELTSLCGDDTMRLLEKNGLTFPFICKTRVAHGTNSHEMAIVFNQEGLNAIQPPCVVQNFINHNAVLYKVFVVGESYTVVQRPSLKNFSAGTSDRESIFFNSHNVSKPESSSVLTELDKIEGVFERPSDEVIRELSRALRQALGVSLFGIDIIINNQTGQHAVIDINAFPGYEGVSEFFTDLLNHIATVLQGQSTAMAATGDVALLRHSKLLAEPAGGLVGERTCSASPGCCGSMMGQDAPWKAEADAGGTAKLPHQRLGCNAGVSPSFQQHCVASLATKASSQ